(19) United States
(12) Patent Application Publication
(10) Pub. No.: US 2018/0369847 A1
(43) Pub. Date: Dec. 27, 2018

(54) Mobile Fragrance Discharge Device

(71) Applicant: Pium Labs, Inc., Fort Lee, NJ (US)

(72) Inventors: Jaeyeon Kihm, Fort Lee, NJ (US); Sung Jae Hwang, Seongnam-si (KR); Jung Ho Kim, Seoul (KR)

(21) Appl. No.: 16/121,309

(22) Filed: Sep. 4, 2018

Related U.S. Application Data
(63) Continuation-in-part of application No. PCT/KR2017/002278, filed on Feb. 3, 2017.

(30) Foreign Application Priority Data
Mar. 4, 2016 (KR) 10-2016-0026674
Jun. 16, 2016 (KR) 10-2016-0075075

(51) Int. Cl. B05B 12/16; B05B 15/60; B05B 12/00

(52) U.S. Cl. CPC B05B 12/16; B05B 12/004; B05B 15/60

(57) ABSTRACT

Provided is a mobile fragrance discharge device. The mobile fragrance discharge device includes a body configured to be movable; a storage mounted to the body and configured to store a fragrance material; a discharge portion configured to connect to the storage and to discharge, to an outside of the body, at least a portion of the fragrance material supplied from the storage; a location detector configured to detect a location or a travel route of the body; and a controller configured to determine a discharge characteristic of the fragrance material based on location information or travel route information of the body and to control the discharge portion to discharge the fragrance material based on the determined discharge characteristic.

This application is related to concurrently filed U.S. application Ser. No. ______, entitled “Fragrance Discharge Device and Control Method Thereof,” and concurrently filed U.S. application Ser. No. ______, entitled “Fragrance Discharge Device,” which are herein incorporated by reference in their entirety for all purposes.

### TECHNICAL FIELD

Embodiments relate to a mobile fragrance discharge device.

### BACKGROUND ART

A fragrance refers to a pleasant odor that may come from flowers, incense, perfume, etc. It has long been known effect that a specific fragrance may affect psychology and emotions of a user. Therefore, people are using perfume for various purposes, such as mood stabilization, psychological stability, and ventilation.

Perfume is generally provided in a liquid form and may be stored and used in a container provided with a device capable of dispensing liquid. In general, perfume in a container is kept at a desired place and used there or by being dispensed by the user over a body part such as a wrist and a neck of the user or clothes of the user as desired.

However, perfume according to the related art is placed in a specific space to spread a fragrance within the space through a natural diffusion method or to discharge a fragrance material at desired intervals.

Such perfume according to the related art may not actively operate. For example, such perfume may not apply spatial characteristics of a specific space, for example, an office, a private room, a kitchen, and a toilet, and, in addition to the spatial characteristics, may not apply a condition of a user.

Accordingly, perfume may be discharged unnecessarily although the user is absent in the specific space, may not be discharged at a proper point in time at which the perfume needs to be discharged, or may be discharged periodically in a situation in which a plurality of persons is present and discharging of a specific fragrance is improper. That is, perfume according to the related art may not suitably meet the potential intent and needs of the user.

In the meantime, since such perfume according to the related art operates at a location fixed within a specific space, a fragrance may not be provided to a user away from the perfume and it may be impossible to actively cope with a situation in which the fragrance needs to be provided at a specific location.

### SUMMARY

Embodiments provide a mobile fragrance discharge device that may control discharging of a fragrance material based on location information or travel route information.

Embodiments also provide a mobile fragrance discharge device that may discharge a fragrance material at a specific location or in a specific section within a travel route while moving to a destination.

Embodiments also provide a mobile fragrance discharge device that may control discharging of a fragrance material within a space in conjunction with an external device.

According to an aspect, there is provided a mobile fragrance discharge device including a body configured to be movable; one or more storages configured to mount to the body and to store a fragrance material; a discharge portion provided to the body and configured to discharge, to an outside, the fragrance material supplied from the storage; a location detector configured to detect a location or a travel route of the body; a controller configured to determine a discharge characteristic of the fragrance material based on location information or travel route information of the body, and to control a fragrance material discharge of the discharge portion based on the determined discharge characteristic; and a propeller provided to the body and configured to provide a driving force so that the mobile fragrance discharge device flies in a three-dimensional (3D) space.

Also, the discharge characteristic may include at least one of whether to discharge the fragrance material, a type, an amount, a discharge direction, and a discharge pattern of the fragrance material to be discharged, a blending ratio, and a discharge location of the fragrance material on a space.

Also, the controller may be configured to control the fragrance material discharge of the discharge portion at a predetermined location or in a predetermined section within the travel route.

Also, the controller may be configured to control the discharge portion by further considering at least one of spatial information associated with a space in which the mobile fragrance discharge device is present, state information of the mobile fragrance discharge device, and information associated with a person or a object present within the space.

Also, the mobile fragrance discharge device may further include a sensor portion provided to the body and configured to sense spatial information including a spatial characteristic around the mobile fragrance discharge device. The controller may be configured to control the discharge portion based on the spatial information.

Also, the sensor portion may include at least one of an image sensor, a pollution measurement sensor, a temperature measurement sensor, a fluid velocity sensor, an illuminance sensor, an odor sensor, a location recognition sensor, a proximity sensor, and a sound sensor.

Also, the mobile fragrance discharge device may be configured to mount to a movable external device and to be movable.

Also, the mobile fragrance discharge device may further include a communicator configured to receive the location or the travel route of the body from the mobile external device. The location detector may be configured to detect the location of the body based on the received location information or travel route information.

Also, the propeller may include one or more rotors, and the mobile fragrance discharge device may be a drone capable of moving and hovering in a 3D space.

Also, the mobile fragrance discharge device may further include a communicator configured to interact with an external device and to communicably connect to the external device to control discharging of the fragrance material.

Also, the location detector may be configured to determine the travel route of the body based on fragrance material related information from the external device.

Also, the fragrance material related information may include at least one of multimedia content information provided from the external device, spatial information including a spatial characteristic around the mobile fragrance discharge device, time information, odor information, user body information of a user of the mobile fragrance discharge device, a relative location between the mobile fragrance discharge device and the user, and preference information of the user on a specific fragrance material.

Also, the controller may be configured to receive a predetermined travel route from the external device through the communicator and to control the propeller so that the body moves along the predetermined travel route.

Also, the controller may be configured to control the propeller to control a travel speed of the mobile fragrance discharge device.

Also, the external device may be a mobile device, and a list of external devices operable in conjunction with the mobile fragrance discharge device or a control user interface for controlling the mobile fragrance discharge device may be provided to the external device.

Also, the external device may be another fragrance discharge device, and the location detector may be configured to determine the travel route on a relative location with the other fragrance discharge device and to control discharging of the fragrance material.

Also, the location detector may be configured to determine the travel route of the body based on fragrance material related information received from the external device, and to determine again the travel route of the body based on new fragrance material related information in response to receiving the new fragrance material related information.

Also, the external device may be a multimedia player and the mobile fragrance discharge device may move a location or control discharging of the fragrance material based on content information of multimedia being played on the external device.

Also, the external device may be a virtual reality (VR) providing device and the mobile fragrance discharge device may move to a location relative to the user or control discharging of the fragrance material based on location information of a user within virtual reality (VR) provided from the VR providing device.

Also, the mobile fragrance discharge device may further include a fan configured to effectively diffuse the fragrance material, and the fan may operate based on the discharge characteristic of the fragrance material.

Also, the mobile fragrance discharge device may further include an air cleaner configured to clean the air around the mobile fragrance discharge device, and the air cleaner may operate based on the discharge characteristic of the fragrance material.

Also, the mobile fragrance discharge device may further include an artificial intelligence (AI) learner configured to learn a control history of the controller on discharging of the fragrance material based on at least one of the spatial information, the state information of the mobile fragrance discharge device, and the information associated with the person or the object present within the space. The controller may be configured to control the discharge portion using a scheme recommended by the AI learner.

Also, the AI learner may be configured to learn the control history using a time series data analysis.

According to an aspect, there is provided a mobile fragrance discharge device communicably connected to an external device capable of discharging one or more fragrance materials, the mobile fragrance discharge device including a body configured to be movable; one or more storages configured to mount to the body and to store a fragrance material a discharge portion provided to the body and configured to discharge, to an outside, the fragrance material provided from the storage; a location detector configured to detect a location or a travel route of the body; a propeller provided to the body and configured to provide a driving force so that the mobile fragrance discharge device flies in a 3D space; a communicator configured to communicably connect to the external device; and a controller configured to determine a discharge characteristic of the fragrance material based on location information or travel route information of the body, and to control the discharge portion to discharge the fragrance material based on the determined discharge characteristic. The controller is configured to control a fragrance material discharge of the external device through the communicator.

A mobile fragrance discharge device according to embodiments may determine a discharge characteristic of a fragrance material based on location information or travel route information and may control discharging of the fragrance material based on the determined discharge characteristic of the fragrance material.

Also, the mobile fragrance discharge device may discharge the fragrance material without a need for a user to directly manipulate the device.

Also, the mobile fragrance discharge device may include a propeller and may discharge the fragrance material at a specific location or in a specific section within a travel route.

Also, the mobile fragrance discharge device may control discharging of the fragrance material within a space in conjunction with an external device.

Also, the mobile fragrance discharge device may economically use the fragrance material by controlling discharging of the fragrance material based on the location information or the travel route information.

### BRIEF DESCRIPTION OF DRAWINGS

FIG. 1 is a block diagram illustrating a configuration of a mobile fragrance discharge device according to an embodiment.

FIG. 2 is a perspective view of the mobile fragrance discharge device including the configuration of FIG. 1 according to an embodiment.

FIG. 3 is a perspective view illustrating a cleaning robot to which the mobile fragrance discharge device of FIG. 2 is mounted.

FIG. 4 is a perspective view of a mobile fragrance discharge device according to another embodiment.

FIG. 5 is a perspective view of a mobile fragrance discharge device according to another embodiment.

FIG. 6 is a block diagram illustrating a configuration of the mobile fragrance discharge device of FIGS. 4 and 5 and an external device interacting therewith.

FIGS. 7 through 10 illustrate examples of a user interface of an external device according to an embodiment.

FIG. 11 is a block diagram illustrating a configuration of the mobile fragrance discharge device of FIG. 6 and another external device interacting therewith.

FIG. 12 is a block diagram illustrating another configuration of the mobile fragrance discharge device of FIG. 6 and another external device interacting therewith.

FIG. 13 is a flowchart illustrating a control method of a mobile fragrance discharge device according to an embodiment.

FIGS. 14 through 18 each is a flowchart illustrating a control method of a mobile fragrance discharge device according to another embodiment.

FIGS. 19 and 20 illustrate examples in which a mobile fragrance discharge device interacts with a multimedia player according to an embodiment.

FIG. 21 illustrate an example in which a mobile fragrance discharge device interacts with another multimedia player according to an embodiment.

FIG. 22 illustrates an example in which a mobile fragrance discharge device interacts with various external devices according to an embodiment.

FIG. 23 illustrates an example of setting a path of a mobile fragrance discharge device according to an embodiment.

FIG. 24 illustrates another example of setting a path of a mobile fragrance discharge device according to an embodiment.

FIG. 25 illustrates an example in which a plurality of mobile fragrance discharge devices operates through mutual interaction according to an embodiment.

FIG. 26 illustrates another example in which a plurality of mobile fragrance discharge devices operates through mutual interaction according to an embodiment.

FIG. 27 is a block diagram illustrating a configuration of a mobile fragrance discharge device according to another embodiment.

FIG. 28 illustrates a learning process of an artificial intelligence (AI) learner of FIG. 27.

### DETAILED DESCRIPTION

Hereinafter, embodiments will be described in detail with reference to the accompanying drawings such that those having ordinary skill in the art to which the present disclosure pertains may easily practice the present disclosure. The present disclosure may, however, be embodied in many different forms and should not be construed as being limited to the embodiments set forth herein. In addition, in the description of the present disclosure, detailed description of well-known related structures or functions will be omitted when it is deemed that such description will cause ambiguous interpretation of the present disclosure.

The terminology used herein is for the purpose of describing particular embodiments only and is not intended to be limiting. As used herein, the singular forms “a,” “an,” and “the,” are intended to include the plural forms as well, unless the context clearly indicates otherwise.

It will further be understood that the terms “comprises,” “comprising,” “includes,” and/or “including,” when used herein, specify the presence of stated features, integers, steps, operations, components, and/or parts, but do not preclude the presence or addition of one or more other features, integers, steps, operations, components, parts, and/or groups thereof.

Further, the following embodiments are provided for further clear explanation for those of ordinary skill in the art and thus, shapes and sizes of elements shown in the drawings may be exaggerated for the clear explanation.

### Configuration of Mobile Fragrance Discharge Device

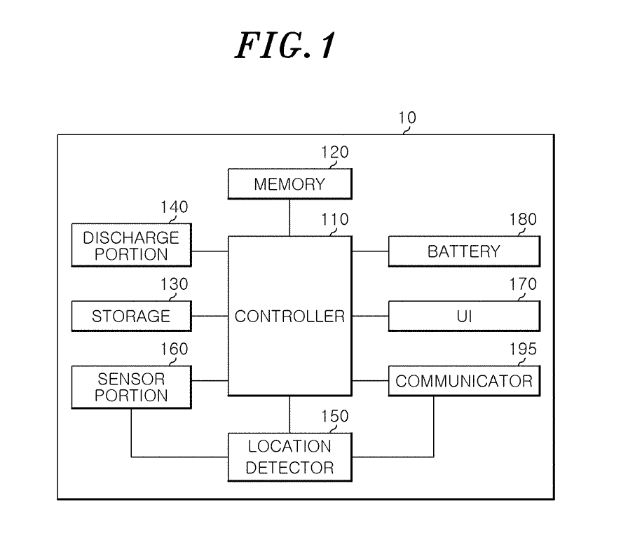

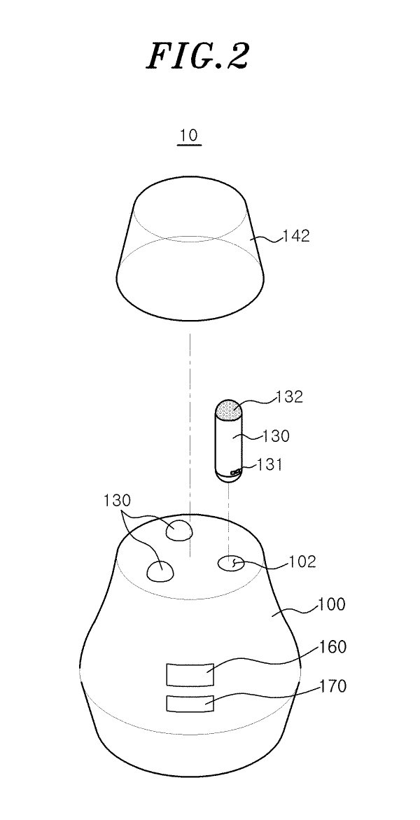

FIG. 1 is a block diagram illustrating a configuration of a mobile fragrance discharge device according to an embodiment and FIG. 2 is a perspective view of the mobile fragrance discharge device including the configuration of FIG. 1 according to an embodiment.

Referring to FIGS. 1 and 2, a mobile fragrance discharge device 10 according to an embodiment is configured to discharge at least one fragrance material stored inside to an outside based on a location or a travel route of a body 100.

In detail, the mobile fragrance discharge device 10 may include a storage 130 configured to store a fragrance material, the body 100 to which one or more storages 130 are mounted and configured to be mobile, a discharge portion 140 provided to the body 100 and configured to discharge, to an outside, the fragrance material supplied from the storage 130, a controller 110 configured to control the discharge portion 140 to discharge, to the outside, the fragrance material stored in the storage 130, and a location detector 150 configured to detect a location or a travel route of the body 100.

Here, the fragrance material refers to a material capable of emitting a fragrance recognizable through an olfactory sense of a person and is not limited to a state of a material stored in the storage 130 or a state of the material discharged through the discharge portion 140. That is, the fragrance material may be stored in at least one of a liquid state, a gas state, and a solid state in the storage 130 of the mobile fragrance discharge device 10. Also, the fragrance used herein may also represent a scent and a smell, etc., and thus may be interchangeably used therewith. For example, the fragrance material may be stored in the liquid state in the storage 130 and may be evaporated and then discharged to the outside of the body 100. Also, the fragrance material may have one or more fragrances. For example, if the fragrance material has a plurality of fragrances, the fragrance material may be a mixture of a plurality of materials each capable of emitting a fragrance.

The body 100 may be accommodated within a predetermined space or may be mounted to a moving object, and has a size and a shape capable of accommodating one or more storages 130, the controller 110, a battery 180, and the like. The body 100 may be formed using a plastic or metal material, and may have a streamlined appearance in a pot shape as illustrated in the figures, and may be in an approximately rectangular box shape. However, it is provided as an example only and the overall shape of the body 100 may be modified if necessary. In the meantime, the body 100 may be disposed in a specific space and used only at a corresponding location, and may be mounted on a moving object such as a vehicle. A lower portion of the body 100 may be in a flat shape to be safely placed within the specific space. Also, the body 100 may include a mounting hole 102 into which each of the one or more storages 130 may insert. An upper cover 142 may be provided on a top of the body 100 to protect the storage 130 and to achieve an aesthetic appearance. The upper cover 142 may have a circular cross-section based on a shape of the top of the body 100, and, without being limited thereto, may have a cross-section in various shapes based on the shape of the body 100. Also, a top of the upper cover 142 may be open to discharge, to the outside, the fragrance material supplied from the storage 130. Also, an entire top surface of the upper cover 142 may be sealed and may include a plurality of discharge holes through which the fragrance material is discharged to the outside. Although not illustrated, a nozzle may be provided to the upper cover 142 and the upper cover 142 may be rotatably mounted to the body 100. Accordingly, a discharge direction of the fragrance material may be controlled through rotation of the upper cover 142. In an embodiment, an actuator (not shown) configured to rotate the upper cover 142 may be provided in the body 100 and the upper cover 142 may automatically rotate under control of the controller 100.

The storage 130 is configured to store a fragrance material in a predetermined state. Herein, the present embodiment is described based on an example in which the fragrance material is stored in a liquid state and the storage 130 is in an elongated-capsule shape to store the fragrance material. However, as described above, the fragrance material may be stored in various states and the storage 130 may be in a shape corresponding thereto. For example, the fragrance material may be provided in a powder form that includes solid particles. In this case, the storage 130 may include a structure, for example, a membrane to which powders may be attached and fastened. Herein, three storages 130 may be provided each in a cartridge form, and may store different fragrance materials, respectively. Each storage 130 may be inserted into each corresponding mounting hole 102 formed in the body 100. Accordingly, once the fragrance material stored in the storage 130 is all used and needs to be replaced, a user may simply replace the corresponding storage 130. The storage 130 may be formed using a material, such as glass and plastic, and may be molded using an anticorrosive material against chemical components of various fragrance materials. Also, the storage 130 may be formed using a transparent material. In this case, although the storage 130 is mounted to the body 100, a remaining amount of the fragrance material may be easily verified through a transparent window (not shown) provided to the body 100. One or more discharge holes 132 each with a minute size may be formed in an upper portion of the storage 130. Accordingly, the fragrance material stored in the storage 130 may be evaporated through the discharge portion 140 and discharged to the outside of the storage 130 through the discharge hole 132. Further, a device capable of controlling an environmental factor, such as a temperature and a humidity, may be provided in the body 100 such that the fragrance material stored in the storage 130 may not be changed and may be stably stored. For example, if an optimal temperature is required to enhance effect of emitting the fragrance material, a temperature maintaining device, such as a thermoelectric element provided in the body 100, may maintain the storage 130 to be in the optimal temperature.

The discharge portion 140 is provided to discharge, to the outside, the fragrance material stored in the storage 130. Here, discharging of the fragrance material may include dispensing the fragrance material to the outside of the mobile discharge device 10 at predetermined pressure by directly applying a physical external force to the fragrance material and naturally emitting the fragrance material without applying the direct external force. For example, spraying the fragrance material using a predetermined device and naturally evaporating the fragrance material may be understood as discharging by the discharge portion 140. Various fragrance materials each stored in the corresponding storage 130 may be discharged to the outside using, for example, an ultrasonic vibration scheme, a natural evaporation scheme, a heating scheme, and a spray scheme. For example, an ultrasonic vibrator (not shown) may be provided in each storage 130 and an electrode portion 131 may be provided on each storage 130. Accordingly, once the storage 130 inserts into the mounting hole 102 of the body 100, an electrode provided to the body 100 and the electrode portion 131 of the storage 130 may be connected to each other to electrically control the ultrasonic vibrator. Although not illustrated herein, when the storage 130 inserts into the mounting hole 102 of the body 100 and then the upper cover 142 is provided to the body 100, an electrode provided to the upper cover 142 may be connected to an electrode provided on an upper side of the storage 130 to electrically control the ultrasonic vibrator. When the storage 130 needs to be replaced due to a lack of an amount of the fragrance material remaining in the storage 130, the controller 110 may control the discharge portion 140 to temporarily suspend discharging of the fragrance material.

The controller 110 may be provided in the body 100. Controller 110 may be any suitable processing device capable of receiving and transmitting digital electronic signals such as, by way of example, a microprocessor, a microcontroller, a digital integrated circuit, etc. Controller 110 may further include or be in communication with a memory 120.

The controller 110 may control the discharge portion 140 based on context information including at least one of user information of a user using the mobile fragrance discharge device 10 and environmental information around the body 100 such that the fragrance material stored in the storage 130 may be discharged to the outside. The controller 110 may control at least one of whether to discharge the fragrance material, a type, an amount, a discharge direction, and a discharge pattern of the fragrance material that is discharged through the discharge portion 140, a blending ratio, and a discharge location of the fragrance material on the space. Here, the user information refers to information associated with a body or a concept of the user and may include at least one of a location of the user within a space in which the mobile fragrance discharge device 10 is present, a relative location between the user and the mobile fragrance discharge device 10, a type of a target, such as a person and an animal, toward which the fragrance material is discharged, an olfactory sensitivity of the user, a preference of the user on a specific fragrance material, and body information such as an emotion, stress, and a concept of the user, a change in an action of the user, and a motion of the user. Also, the environmental information may refer to information on an environment around a space in which the mobile fragrance discharge device 10 is present, and may include at least one of characteristics of the space in which the mobile fragrance discharge device 10 is present, for example, an ideal concept of space including a virtual space and a physical space such as a room, an office, a closet, a vehicle, and an airplane, illuminance, odor information, ventilation information, time information, air pollution information, and weather information.

The controller 110 determines a discharge characteristic of the fragrance material based on location information of the mobile fragrance discharge device 10 or travel route information of the body 100 and controls the discharge portion 140 to discharge the fragrance material to the outside based on the determined discharge characteristic. Here, the discharge characteristic may include at least one of whether to discharge the fragrance material, a type, an amount, a discharge direction, and a discharge pattern of the fragrance material to be discharged, and a blending ratio for a plurality of fragrance materials, and a discharge location of the fragrance material in a space. The controller 110 may control the fragrance material discharge of the discharge portion 140 at a predetermined location or in a predetermined section within the travel route through which the body 100 moves. Also, the controller 110 may control the fragrance material discharge of the discharge portion 140 by further considering at least one of spatial information associated with a space in which the body 100 is present, state information of the mobile fragrance discharge device 10, and information associated with a person or an object within the space in which the mobile fragrance discharge device 10 is present. Description related to various embodiments in which the mobile fragrance discharge device 10 operates based on the context information, the location information, and the travel route information is further described below.

The mobile fragrance discharge device 10 according to an embodiment may further include the location detector 150. The location detector 150 may detect a current location of the mobile fragrance discharge device 10 or may detect a travel route of the mobile fragrance discharge device 10. The location detector 150 may verify the current location using a global positioning system (GPS) sensor, or may verify a location of the mobile fragrance discharge device 10 within the space using at least one of a gyro sensor, an acceleration sensor, and a speed sensor. Also, the location detector 150 may receive image information sensed at a sensor portion 160, may analyze received environmental information around the space, for example, size and structure information of the space, and may generate information on the location of the mobile fragrance discharge device 10 within the space. Also, the location detector 150 may directly verify the location within the space using an ultrasonic sensor, an IR sensor, a recognition sensor, or a camera. For example, the location detector 150 may use a gyro sensor, an acceleration sensor, or a speed sensor to detect an accurate location within a specific indoor space and may detect its location using a GPS sensor in a relatively wide outdoor space.

In the meantime, when the mobile fragrance discharge device 10 communicably connects to another external device such as a smartphone or a wearable device of the user, the mobile fragrance discharge device 10 may directly receive location information or travel route of the mobile fragrance discharge device 10 from the external device and may verify the location or the travel route. Location information of the body 100 detected by the location detector 150, that is, location information or travel route information of the mobile fragrance discharge device 10 may be transmitted to the controller 110. The controller 110 may determine the discharge characteristic of the fragrance material based on the received location information or travel route information and may control the fragrance material discharge of the discharge portion 140 based on the determined discharge characteristic. Although the present embodiment is described based on an example in which the location detector 150 and the controller 110 are separately provided, the location detector 150 may be provided in the controller 110 as a functional unit.

The mobile fragrance discharge device 10 according to an embodiment may further include the sensor portion 160. The sensor portion 160 is configured to actively detect environmental information around the space in which the mobile fragrance discharge device 10 is present and may include at least one of an image sensor, a pollution measurement sensor, a temperature measurement sensor, a fluid velocity sensor, an illuminance sensor, an odor sensor, a proximity sensor, a sound sensor, an ultrasonic sensor, an IR sensor, an image sensor, and a camera. The sensor portion 160 may transmit the sensed information to the controller 110 or the location detector 150, and the controller 110 may control discharging of the fragrance material by controlling the discharge portion 140 based on the received environmental information. For example, the sensor portion 160 may be an image sensor or an IR sensor, and may recognize a target around the mobile fragrance discharge device 10 and may transmit corresponding information to the controller 110 or the location detector 150. The location detector 150 may analyze the received environmental information, for example, size and structure information of the space, and may generate information on the location of the mobile fragrance discharge device 10 within the space, and may transmit the generated information to the controller 110. The controller 110 may control the discharge portion 140 based on attribute information or location information of the target. Therefore, when a person or an object is present within a predetermined distance from the mobile fragrance discharge device 10, the controller 110 may control the discharge portion 140 to temporarily stop discharging of the fragrance material or to adjust a discharge direction of the fragrance material, so that the fragrance material may not be directly discharged toward the person or the object. Also, the controller 110 may adjust a distance, intensity, or interval at which the fragrance material is discharged so that the fragrance material may not be directly discharged toward the person or the object. Also, when the space in which the mobile fragrance discharge device 10 is present is unsuitable for discharging the fragrance material based on an air environment condition, such as that wind is measured to be very strong at the fluid velocity sensor or that air pollution is sensed to be serious at the air pollution sensor, the controller 110 may adjust the fragrance material to not be discharged. When the mobile fragrance discharge device 10 moves to a new location at which discharging of the fragrance material is suitable, the controller 110 may adjust the fragrance material to be discharged.

The mobile fragrance discharge device 10 according to an embodiment may further include a user interface (UI) 170 provided to the body 100. The UI 170 may be provided in a form of a touch display device or a light emitting diode (LED) display on the outer surface of the body 100. Thus, if a remaining amount of the fragrance material is insufficient, the mobile fragrance discharge device 10 may provide an alert notification to the user through the UI 170. Also, if the remaining amount of the fragrance material is insufficient, the mobile fragrance discharge device 10 may inform the user of the insufficiency through an alert sound. Also, the mobile fragrance providing device 10 may directly receive an input from the user through a touch display device to operate in a predetermined mode. For example, the user may select a desired fragrance material through the UI 170. Also, the UI 170 may display a type of a mode currently in operation, information on the fragrance material, and a scheduled replacement time. When a plurality of storages 130 is provided, the UI 170 may display a remaining amount of a fragrance material of each storage 130 and may also display a remaining amount acquired by adding up amounts of fragrance materials remaining in the respective storages 130. If the mobile fragrance discharge device 10 malfunctions, the UI 170 may display corresponding error information.

The memory 120 may store a variety of information sensed at the sensor portion 160, information exchanged with the external device through a communicator 195, and data input from the user and data provided to the user through the UI 170. For example, the memory 160 may store user information including motion information of the user and biometric information of the user, location information, travel route information, environmental information, state information of the mobile fragrance discharge device 10, and discharge information of the fragrance material.

The mobile fragrance discharge device 10 according to an embodiment may further include the battery 180. The battery 180 may supply power required to operate the controller 110, the discharge portion 140, the location detector 150, the sensor portion 160, and the UI 170. The battery 180 may use all of a replaceable battery and a chargeable battery. When the replaceable battery is used for the battery 180, a cover may be provided to the body 100 to replace the battery 180. When the chargeable battery is used for the battery 180, a connection terminal for charging may be provided to the body 100. Also, the battery 180 may be a wirelessly chargeable battery. Here, wireless charging refers to charging the battery 180 without using a terminal exposed to an outside. Various wireless charging techniques may be applied to the mobile fragrance discharge device 10. For example, the battery 180 may be charged using an electromagnetic induction scheme. The electromagnetic induction scheme refers to a scheme of using an electromagnetic induction principle in which, if an electromagnetic field is generated in a power transmitting coil, electricity is induced from a power receiving coil due to effect of the magnetic field. In detail, the battery 180 may include an iron core. If the battery 180 is around a wireless charging device with alternating current (AC), charging may be performed by a constantly changing magnetic field. Alternatively, the battery 180 may be wirelessly charged using a magnetic resonance scheme or an electromagnetic wave scheme. The electromagnetic wave scheme refers to a scheme of receiving, by a receiver, the electromagnetic wave using a rectenna in which an antenna and a rectifier are combined and converting the received electromagnetic wave to power in response to the electromagnetic wave being generated in a transmitter. Also, the electromagnetic resonance scheme refers to technology that allows a transmitting coil to generate a magnetic field vibrating at a resonant frequency so that energy may be concentratively transferred only to a receiving coil designed based on the same resonant frequency. Ampere's law and Faraday's law are commonly applied to the aforementioned electromagnetic induction scheme, electromagnetic wave scheme, and electromagnetic resonance scheme. Here, wireless charging of the battery 180 may be performed by a separately provided charger, for example, a docking device, and may be performed through the external device 20, which is described below. In detail, a charging device configured to generate a magnetic field may be provided to the external device 20, so that the aforementioned wireless charging may be performed at a location corresponding to a location at which the mobile fragrance discharge device 10 is mounted. Also, the battery 180 may be a solar cell. In this case, a light collector, for example, a PN junction semiconductor, capable of collecting sunlight may be provided on an outer surface of the body 100. Alternatively, the light collector may be provided to the external device 20 and the battery 180 may be electrically connected to the light collector of the external device 20.

The mobile fragrance discharge device 10 according to an embodiment may further include the communicator 195. The communicator 195 is provided such that the mobile fragrance discharge device 10 may control discharging of a fragrance material in conjunction with a smartphone, a multimedia player, or another external device capable of discharging the fragrance material. For example, the mobile fragrance discharge device 10 may connect to a mobile device, such as a smartphone of the user, through the communicator 195 and the user may control fragrance material discharge of the mobile fragrance discharge device 10 by controlling the controller 110. Also, the mobile fragrance discharge device 10 may recognize environmental information around the space in which the mobile fragrance discharge device 10 is installed, location information, and user information including biometric information of the user using various sensors mounted to the mobile device, and may transmit the recognized information to the controller 110 through the communicator 195. The mobile fragrance discharge device 10 may connect to the external device 20 in a wired or wireless manner and may communicate therewith using one of, for example, a universal serial bus (USB), Infrared Data Association (IrDA), wireless fidelity (WiFi), ultra-wideband (UWB), Bluetooth, ZigBee, code division multiple access (CDMA), wideband code division multiple access (WCDMA), Long Term Evolution (LTE), and LTE-Advanced (LTE-A). That is, the mobile fragrance discharge device 10 may connect to the external device 20 in a wired manner using a USB, may directly connect thereto in a wireless manner through WiFi, and may also connect thereto in a wireless manner through an access point (AP).

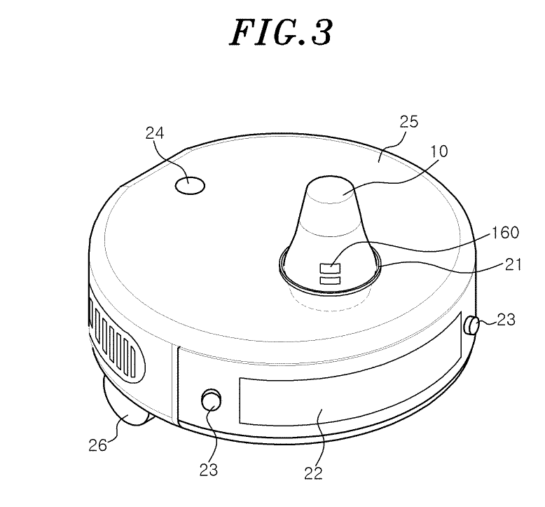

FIG. 3 illustrates an example in which the mobile fragrance discharge device of FIG. 2 is mounted to the mobile external device.

Referring to FIG. 3, the mobile fragrance discharge device 10 according to an embodiment may be mounted to a movable mobile external device and may move together therewith. The present embodiment is described using a robot cleaner 25 as an example of the mobile external device. The robot cleaner 25 includes a disk-shaped body, a dust suction chamber 22 configured to collect dust in a cleaning area, a detector 23 configured to detect the cleaning area and an obstacle, and a moving wheel 26 as a driving device for driving. The robot cleaner 25 sucks foreign substances such ad dust in the cleaning area using the dust suction chamber 22 and automatically cleans the cleaning area while autonomously driving within an area to be cleaned. The detector 23 is configured to verify the cleaning area in a space in which the robot cleaner 25 operates and to recognize an obstacle within the space, and may include a location sensor, an angular velocity sensor, a speed sensor, an acceleration sensor, a proximity sensor, a torque sensor, an IR sensor, an ultrasonic sensor, an image sensor, and a camera. The robot cleaner 25 may divide the cleaning area into a plurality of areas based on spatial information recognized by the detector 23 and may set a travel route through which the robot cleaner 25 sequentially moves each area. The robot cleaner 25 cleans around while moving along the travel route. A mounter 21 to which the mobile fragrance discharge device 10 is mountable may be provided on a top surface of the robot cleaner 25. While the robot cleaner 25 moves along the travel route, the mobile fragrance discharge device 10 may sense environmental information around the space, for example, size and structure information of the space through the sensor portion 160. The sensed environmental information may be transmitted to the location detector 150. The location detector 150 may generate location information of the mobile fragrance discharge device 10 within the space based on the environmental information. The generated location information may be transmitted to the controller 110 of the mobile fragrance discharge device 10, and the controller 110 may determine the discharge characteristic based on the location information and may control discharging of the fragrance material by controlling the discharge portion 140.

Meanwhile, the communicator 195 of the mobile fragrance discharge device 10 may communicably connect to the robot cleaner 25. For example, a communication terminal (not shown) for connection to the external device in a wired manner may be provided on a bottom surface of the mobile fragrance discharge device 10. Therefore, when the mobile fragrance discharge device 10 inserts into the mounter 21 of the robot cleaner 25, the mobile fragrance discharge device 10 may connect to the communication terminal (not shown) provided in the mounter 21 and communication may be connected therebetween. In this case, current location information and travel route information of the robot cleaner 25 verified through the detector 23 of the robot cleaner 25 may be transmitted to the location detector 150 of the mobile fragrance discharge device 10. The location detector 150 may detect the location and the travel route of the mobile fragrance discharge device 10 based on the received location information and travel route information. The controller 110 of the mobile fragrance discharge device 10 may determine a discharge characteristic based on the location information or the travel route information detected by the location detector 150 and may control discharging of the fragrance material by controlling the discharge portion 140. Inversely, the sensor portion 160 of the mobile fragrance discharge device 10 may be recognize an environment around the mobile fragrance discharge device 10 and the location detector 150 may set a travel route based on characteristic information of the recognized space. The travel route may be transmitted to the robot cleaner 25 and the robot cleaner 25 may move along the received travel route.

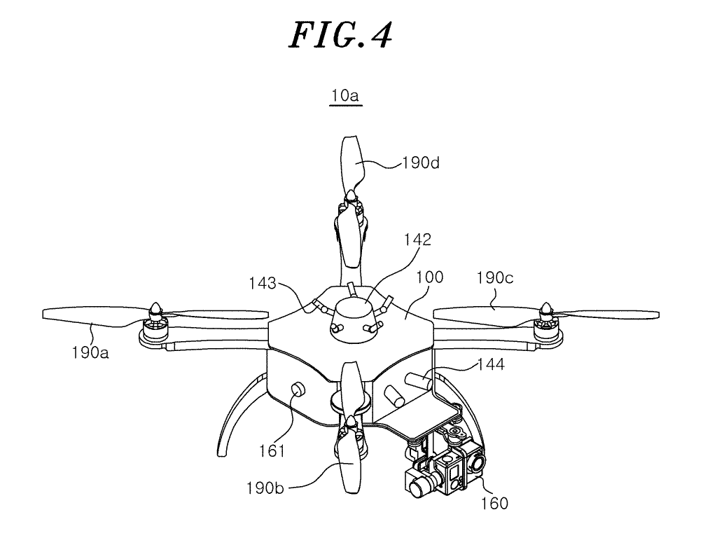

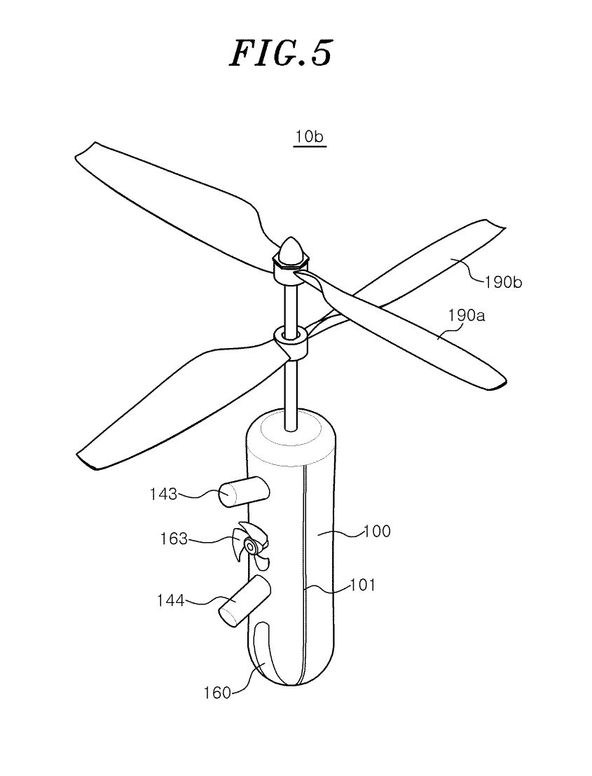

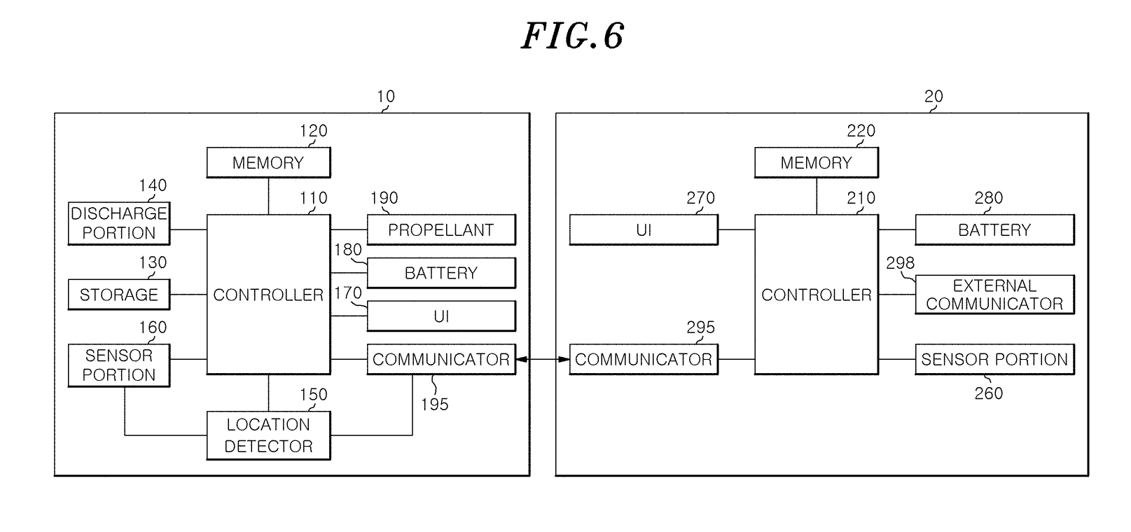

FIGS. 4 and 5 are perspective views illustrating a mobile fragrance discharge device according to another embodiment. FIG. 6 is a block diagram illustrating a configuration of the mobile fragrance discharge device shown in FIGS. 4 and 5 and an external device interacting therewith.

Referring to FIGS. 4 and 6, a mobile fragrance discharge device 10a according to another embodiment may be a drone capable of flying in a space. The mobile fragrance discharge device 10a includes all of the components of the mobile fragrance discharge device 10 of FIG. 1. Thus, only a difference between the present embodiment and the aforementioned embodiment is described here. A plurality of fragrance material storages (not shown) may be replaceably mounted within the body 100 of the drone. The upper cover 142 may be provided on a top of the body 100 to protect the storage 130 provided below the upper cover 142. The upper cover 142 may have a circular cross-section, and, without being limited thereto, may have a cross-section in various shapes. A plurality of upper nozzles 143 each configured to discharge each corresponding fragrance material stored in the storage 130 may be provided to the upper cover 142. Each of the upper nozzles 143 may be mounted to be vertically and horizontally rotatable relative to the upper cover 142 through an actuator (not shown) and the like. Accordingly, although the mobile fragrance discharge device 10a performs hovering at a specific location, the fragrance material may be discharged in various directions. A top of the upper cover 142 may be open to discharge, to the outside, the fragrance material stored in the storage 130 right below the upper cover 142. Also, an entire top surface of the upper cover 142 may be sealed and may include a plurality of discharge holes for discharging the fragrance material to the outside. A lower nozzle 144 for discharging the fragrance material may be provided on at least one side of the body 100. In this case, the upper nozzle 143 may be used to precisely discharge a small amount of fragrance material toward a specific target and the lower nozzle 144 may be used to discharge a large amount of fragrance material over a relatively wide area. Conversely, the upper nozzle 143 may be used to discharge a large amount of fragrance material and the lower nozzle 144 may be used to precisely discharge a small amount of fragrance material. An air cleaner 161 for cleaning the air around the mobile fragrance discharge device 10a may be provided to the body 100. The air cleaner 161 may operate based on the discharge characteristic of the fragrance material. When the air pollution sensed through the pollution measurement sensor of the sensor portion 160 is serious, the mobile fragrance discharge device 10a may operate the air cleaner 161 to clean the ambient air. Also, when the air cleaner 161 serves to clean the air, the air cleaner 161 may be provided at a location at which ventilating air current does not affect controlling the flight of the mobile fragrance discharge device 10. The air cleaner 161 may adjust a direction or intensity for cleaning the air and may include a replaceable filter.

The mobile fragrance discharge device 10a may include a propeller 190 configured to provide a driving force for moving in the space. The propeller 190 may be provided to the body 100 to move the body 100 and may include at least one of a moving wheel, a rotor, and a fixed wing. In the present embodiment, the mobile fragrance discharge device 10a may be a quad-drone that includes four rotors 190a, 190b, 190c, and 190d as the propeller 190. By including the plurality of rotors 190a, 190b, 190c, and 190d, the body 100 may fly in the space and may perform hovering of maintaining a specific location within the space for a relatively long period of time. Accordingly, the mobile fragrance discharge device 10a may discharge a fragrance material by quickly moving to a destination and by precisely aiming at a specific part of the user present at the destination, for example, an olfactory organ of the user. The controller 110 may control a travel speed of the mobile fragrance discharge device 10a by controlling the propeller 190. In the meantime, the mobile fragrance discharge device 10a may include a camera as the sensor portion 160 and may verify a spatial characteristic and a user characteristic through the camera. Through the location detector 150, the mobile fragrance discharge device 10a may use a gyro sensor, an acceleration sensor, or a speed sensor to precisely detect a location within a specific indoor space and may detector its location using a GPS sensor in a relatively wide outdoor area. Also, the mobile fragrance discharge device 10a may sense environmental information around the space, for example, size and structure information of the space through the sensor portion 160 and the sensed environment information may be transmitted to the location detector 150. The location detector 150 may generate location information of the mobile fragrance discharge device 10a within the space based on the environmental information. The generated location information may be transmitted to the controller 110 of the mobile fragrance discharge device 10a. The controller 110 may determine the discharge characteristic based on the location information and may control discharging of the fragrance material by controlling the discharge portion 140.

In the meantime, when the mobile fragrance discharge device 10a communicably connects to the external device 20 of FIG. 6, the mobile fragrance discharge device 10a may directly receive location information or travel route information of the mobile fragrance discharge device 10a from the external device 20 and may verify a location or a travel route. Location information of the body 100 detected by the location detector 150, that is, location information or travel route information of the mobile fragrance discharge device 10 may be transmitted to the controller 110. The controller 110 may determine the discharge characteristic of the fragrance material based on the received location information or travel route information and may control the discharge portion 140 to discharge the fragrance material based on the determined discharge characteristic.

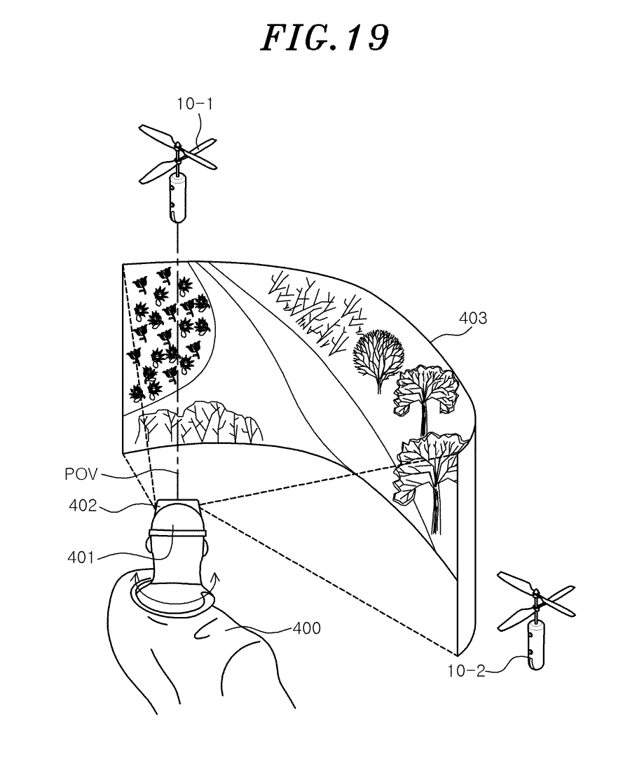

A mobile fragrance discharge device 10b according to another embodiment will be described with reference to FIGS. 5 and 6. The mobile fragrance discharge device 10b includes all of the components of the mobile fragrance discharge device 10 of FIG. 1. Thus, only a difference between the present embodiment and the aforementioned embodiment is described here. A plurality of fragrance material storages (not shown) may be replaceably mounted within the body 100 of the drone. The body 100 has a long cylindrical shape and is relatively small compared to the mobile fragrance discharge device 10a of FIG. 4. The body 100 may be separable into the left and the right based on a coupler 101 and combinable, which enables the storage 130 mounted in the body 100 to be replaceable. The upper nozzle 143 for discharging each corresponding fragrance material stored in the storage 130 may be provided in an upper portion of the body 100. The upper nozzle 143 may be mounted to be vertically and horizontally rotatable relative to the body 100 through an actuator (not shown) and the like. Accordingly, although the mobile fragrance discharge device 10b performs hovering at a specific location, the fragrance material may be discharged in various directions relative to the body 100. The lower nozzle 144 for discharging the fragrance material stored in the storage 130 may be provided in a lower portion of the body 100. Also, the lower nozzle 144 may be mounted to be vertically and horizontally rotatable relative to the body 100 through the actuator (not shown). Accordingly, although the mobile fragrance discharge device 10b performs hovering at a specific location, the fragrance material may be discharged in various directions relative to the body 100. For example, the upper nozzle 143 may be used to precisely discharge a small amount of fragrance material toward a specific target and the lower nozzle 144 may be used to discharge a large amount of fragrance material over a relatively wide area. Conversely, the upper nozzle 143 may be used to discharge a large amount of fragrance material and the lower nozzle 144 may be used to precisely discharge a small amount of fragrance material. A fan 163 for effectively diffusing the fragrance material around the space may be provided to the body 100 between the upper nozzle 143 and the lower nozzle 144. A rotational speed of the fan 163 may be determined based on the discharge characteristic of the fragrance material. In addition to the fan 163, the mobile fragrance discharge device 10b may rotate or move to diffuse the fragrance material toward a specific locational point or a specific direction. Also, by operating the fan 163, it is possible to provide wind strength and temperature directly to a body part of the user or suitable for an area around the body part of the user based on multimedia content provided from the external device 20 of FIG. 6, for example, a head mounted device (HMD) 402 of FIG. 19 worn by the user.

The mobile fragrance discharge device 10b may include two rotors 190a and 190b as the propeller 190 for moving the body 100. By including the plurality of rotors 190a and 190b, the body 100 may fly in the space or may perform hovering at a specific location within the space for a relatively long period of time. Also, the two rotors 190a and 190b vertically provided in the upper portion of the body 100 may control a torque by rotating in mutually reverse directions. That is, with using two rotors instead of using four rotors, the body 100 may stably perform hovering at a constant location through backlash. Also, a size of the mobile fragrance discharge device 10b is miniaturized, thereby reducing the occurrence of rotor-caused noise. Accordingly, a plurality of mobile fragrance discharge devices 10b may interact with each other and may perform a cluster operation. The mobile fragrance discharge device 10b may include a camera or an image sensor as the sensor portion 160 and may verify a spatial characteristic and a user characteristic through the sensor portion 160. Through the location detector 150, the mobile fragrance discharge device 10b may use a gyro sensor, an acceleration sensor, or a speed sensor to precisely detect a location within a specific indoor space and may detect its location using a GPS sensor in a relatively wide outdoor space. Also, the mobile fragrance discharge device 10b may communicably connect to the external device 20 of FIG. 6, may receive location information or travel route information of the mobile fragrance discharge device 10b from the external device 20, and may verify a location or a travel route. Location information of the body 100 detected by the location detector 150, that is, location information or travel route information of the mobile fragrance discharge device 10b may be transmitted to the controller 110. The controller 110 may determine the discharge characteristic of the fragrance material based on the received location information or travel route information, and may control the fragrance material discharge of the discharge portion 140 based on the determined discharge characteristic.

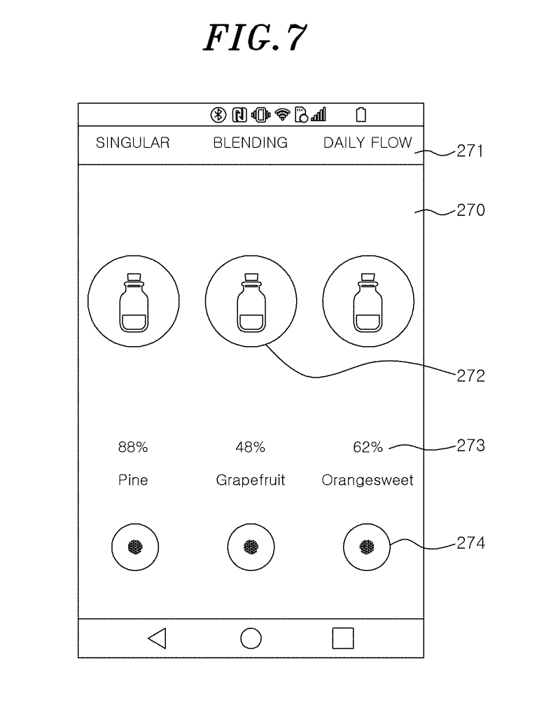

FIGS. 7 through 10 illustrate examples of a user interface of an external device according to an embodiment.

Hereinafter, an interaction between the mobile fragrance discharge device 10 and the external devices 20 is further described.

Referring to FIG. 6, the external device 20 according to an embodiment may be a mobile device such as a smartphone of a user. The external device 20 may include a UI 270 configured to receive user information, a sensor portion 260 configured to recognize environmental information around the external device 20 or the user information, a communicator 295 configured to communicate with the communicator 195 of the mobile fragrance discharge device 10, a controller 210 configured to control the UI 270 and the communicator 295, a memory 220 configured to store data, and an external communicator 298 configured to connect to the Internet or communicate with another external device.

The sensor portion 260 is configured to detect body information of the user or the environmental information around the external device 20 and may include at least one of a location sensor, an odor sensor, a heartrate sensor, a proximity sensor, an illuminance sensor, a temperature sensor, a sound sensor, an IR sensor, and an image sensor. Thus, the mobile fragrance discharge device 10 may use environmental information around a space in which the mobile fragrance discharge device 10 is present, for example, a location and a size of the space, a location of the user, weather, illuminance, temperature, and an odor, and information associated with psychology, a heartrate, and a body of the user, in addition to information measured at the sensor portion 160 of the mobile fragrance discharge device 10. The mobile fragrance discharge device 10 may be controlled based on user profile information stored in the memory 220 of the external device 20. For example, the external device 20 may store, in the memory 220 as a database, a preference of the user on a specific fragrance material, an olfactory sensitivity, and body information such as a biorhythm, and may and may generate user information by combining the information. Also, the generated user information may be stored in a cloud server in addition to the memory 220 of the external device 20. Thus, other user devices communicably connectable to the mobile fragrance discharge device 10 may use the mobile fragrance discharge device 10 based on the user information.

The sensor portion 260 may include a sensor capable of verifying a location, such as a GPS sensor, a gyro sensor, an acceleration sensor, and a speed sensor. The external device 20 may verify current location information through the sensor portion 260 and may set a travel route of the mobile fragrance discharge device 10. The verified location information or set travel route information may be transmitted to the mobile fragrance discharge device 10 through the communicator 295. Also, the external device 20 may connect to an Internet network through the external communicator 298 or another external device, and may receive a current location of the user and a travel route of the mobile fragrance discharge device 10 stored in advance by the user. The received location information and travel route information may be transmitted to the mobile fragrance discharge device 10 through the communicator 295.

The external device 20 may store, in the memory 220, at least one of user information of the user using the mobile fragrance discharge device 10, location information, travel route information, and environmental information around the mobile fragrance discharge device 10 detected through the sensor portion 260, and, if necessary, may transmit corresponding information to the mobile fragrance discharge device 10 through the communicator 250. The controller 110 of the mobile fragrance discharge device 10 may determine the discharge characteristic of the fragrance material based on at least one of the user information, the location information, the travel route information, and the environmental information received from the external device 20 through the communicator 195, and may control the discharge portion 140 based on the determined discharge characteristic.

The controller 210 is provided in a body of the external device 20 and may include, for example, a microprocessor. The controller 210 may control the sensor portion 260, the UI 270, and the communicator 295 of the external device 20. Also, a battery 280 may supply necessary power to the controller 210, the communicator 295, the sensor portion 260, and the UI 270.

If a remaining amount of the fragrance material stored in the storage 130 of the mobile fragrance discharge device 10 is insufficient, the mobile fragrance discharge device 10 may transmit an alert notification indicating the insufficiency of the remaining amount through the communicator 195 and the mobile external device 20 may receive the alert notification through the communicator 295. The received alert notification may be displayed through the UI 270 of the external device 20. Accordingly, the user may opportunely become aware of fragrance material replacement information of the mobile fragrance discharge device 10. Also, the user may control the mobile fragrance discharge device 10 at a remote location through the external device 20. For example, when the user is to move from an outside to a room in which the mobile fragrance discharge device 10 is installed, the user may control the mobile fragrance discharge device 10 to discharge a desired fragrance material in advance through the UI 270 of the external device 20, such that the room may have a fresh atmosphere. Also, the user may directly control a movement of the mobile fragrance discharge device 10 through the external device 20. That is, the user may set a travel route of the mobile fragrance discharge device 10 or may directly move the mobile fragrance discharge device 10 vertically and horizontally using a control device displayed on the UI 270 of the device 20.

Although the present embodiment is described using the smartphone of the user as an example of the external device 20, it is provided as an example only. The external device 20 may be a multimedia player, for example, a wearable device such as a smart band, another mobile device such as a tablet PC, an MP3 player, a personal digital assistant (PDA), a virtual reality (VR) providing device such as an HMD, and smart glasses. In this case, the mobile fragrance discharge device 10 may control discharging of a fragrance material based on information on content being played on the multimedia player, for example, a scene of movie, lyrics, rhythm, and mood of music.

The UI 270 may be configured on a touch display of the external device 20 in response to an operation of an application executed on the external device 20. The UI 270 may provide an alert notification to the user if a remaining amount of a fragrance material of the mobile fragrance discharge device 10 is insufficient, and the user may perform an input through the UI 270 so that the mobile fragrance discharge device 10 may operate in a predetermined mode. For example, the user may select a desired fragrance material through the UI 270. Also, the UI 270 may display information on a fragrance material and a type of a mode currently in operation, and a scheduled replacement period. If the mobile fragrance discharge device 10 malfunctions, the UI 270 may display corresponding error information.

Referring to FIG. 7, the UI 270 of the external device 20 capable of interacting with a fragrance discharge device according to an embodiment is illustrated. The external device 20 refers to a smartphone of a user and includes all of the components of the external device 20 of FIG. 6. The UI 270 refers to an interface for an application executed on the smartphone of the user and the user may control the mobile fragrance discharge device 10 using the UI 270. The UI 270 may include a fragrance material cartridge indicator 272 configured to mount to the mobile fragrance discharge device 10, a remaining amount indicator 273 configured to indicate a remaining amount of a fragrance material, and a control setter 274 configured to set a control environment for discharging each fragrance material. In addition, although not illustrated, the UI 270 may provide an interface capable of displaying activity and biometric information of the user, emotion information, and the like. Through the UI 270, the user may select a setting for a single fragrance material, may select a setting for a blending method thereof, or may select a setting of the mobile fragrance discharge device 10 based on a physical condition or an emotional state of the user according to a daily schedule of the user from a main menu 271.

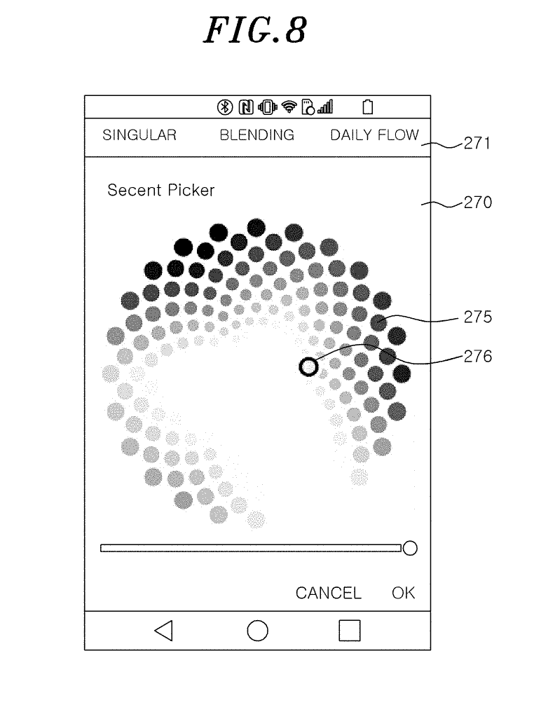

Referring to FIG. 8, the user may effectively select a desired blending ratio 276 through a mixing ratio selection window 275 on which blending ratios 276 for a plurality of fragrance materials are visually displayed. Areas representing two or more fragrance materials may be visually distinguishably displayed on the mixing ratio selecting window 275. A color in which colors representing the respective fragrance materials are mixed based on a ratio of each corresponding fragrance material may be displayed in a neighboring area between different fragrance materials. A ratio of each fragrance material may be calculated in response to the user clicking on a color representing the desired blending ratio 276. The calculated ratio of each fragrance material may be transmitted to the mobile fragrance discharge device. The mobile fragrance discharge device 10 may generate a fragrance desired by the user by selecting a suitable fragrance material based on the received ratio of each fragrance material and by controlling a discharge ratio thereof.

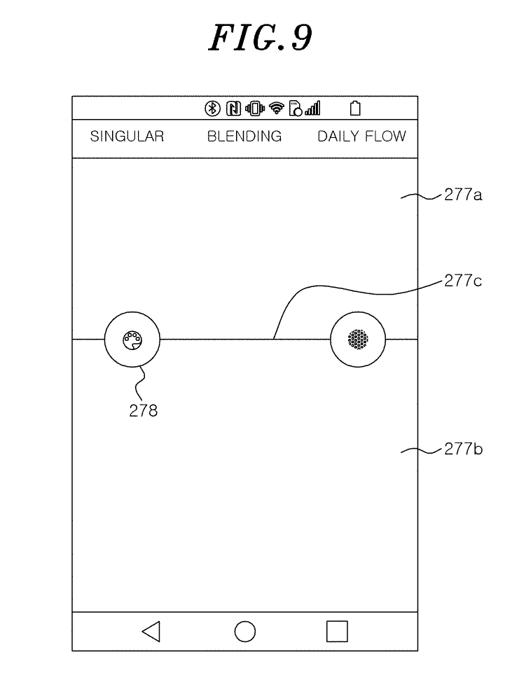

Referring to FIG. 9, the user may select blending ratios for a plurality of fragrance materials through another interface screen. For example, based on a partition line 277c, a surface area of an upper area 277a may represent a relative amount of a first fragrance material and a surface area of a lower area 277b may represent a relative amount of a second fragrance material. The user may adjust a blending ratio of a desired fragrance material by clicking on and vertically moving the partition line 277c. Also, the user may select a fragrance material to be blended by clicking on a fragrance material selection button 278.

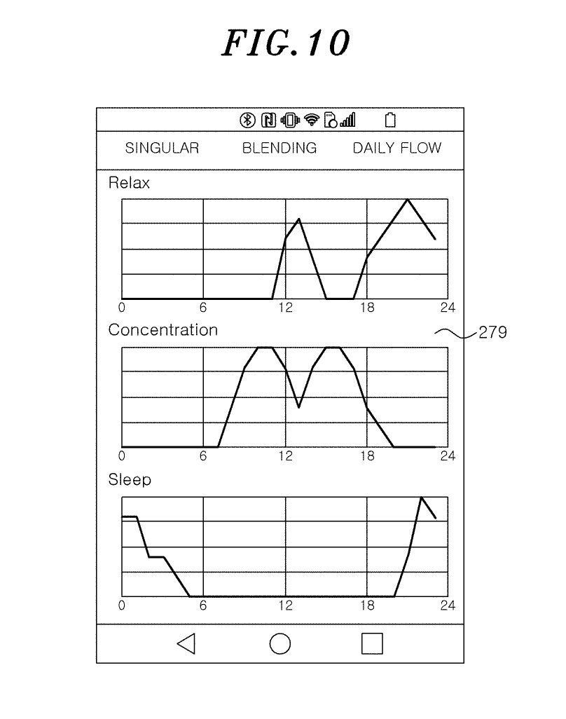

Referring to FIG. 10, information on a physical condition or an emotional state of the user according to a daily schedule may be displayed on a smartphone display 279 of the user. Information on the physical condition or the emotional state of the user measured using various types of sensors mounted to the smartphone of the user. That is, information on an amount of time used to take a rest during the day and a state of the rest, an amount of time used to concentrate on a work during the day and strength of work, and an amount of time used to have a sleep during the day and a state 1 of the sleep may be stored and displayed. The user may set an operating time of the mobile fragrance discharge device 10 based on the above information and may set a discharge point in time, a discharge pattern, and a blending type of a fragrance material such that the mobile fragrance discharge device 10 may provide an olfactory environment suitable for the user.

Although not illustrated, the user may receive a list of external devices operable in conjunction with the mobile fragrance discharge device 10 through another interface screen. Also, the user may view a current location of the mobile fragrance discharge device 10 and may set a destination and a travel route using map information.

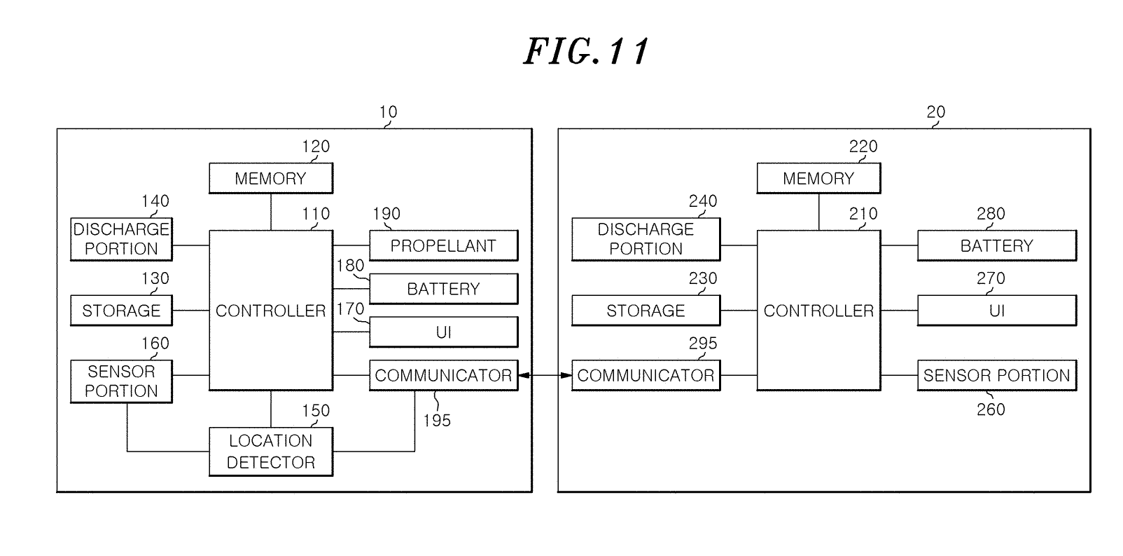

FIG. 11 is a block diagram illustrating a configuration of the mobile fragrance discharge device of FIG. 6 and another external device interacting therewith

Referring to FIG. 11, an external device 20a according to an embodiment refers to a device capable of discharging a fragrance material and is configured to effectively discharge the fragrance material within a predetermined space in conjunction with the mobile fragrance discharge device 10.

In detail, the external device 20a may include a storage 230 configured to store a fragrance material, a body to which one or more storages 230 are mounted, a discharge portion 240 provided to the body and configured to discharge, to an outside, the fragrance material supplied from the storage 230, the controller 210 configured to control the discharge portion 240 to discharge, to the outside, the fragrance material stored in the storage 230, the UI 270 configured to receive user information, the sensor portion 260 configured to recognize environmental information around the external device 20a or the user information, and the communicator 295 configured to communicate with the communicator 195 of the mobile fragrance discharge device 10. A primary configuration of the external device 20a is substantially similar to a function and role of the mobile fragrance discharge device 10 of FIG. 1. Therefore, further description related to the external device 20a is omitted herein and an interaction between the external device 20a and the mobile fragrance discharge device 10 is further described.

The mobile fragrance discharge device 10 and the external device 20a capable of interacting with the mobile fragrance discharge device 10 and discharging a fragrance material may be provided within a predetermined space in which the user is present. In this case, the mobile fragrance discharge device 10 may be movable within the space and the external device 20a may be fixed at a specific location. In this case, the mobile fragrance discharge device 10 and the external device 20a may interact with each other based on information on at least one of in-space location information, travel route information, a relative distance from the user, and body information of the user. For example, when the user is relatively close to the mobile fragrance discharge device 10 and relatively away from the external device 20a, the mobile fragrance discharge device 10 and the external device 20a may verify distances from the mobile fragrance discharge device 10 and the external device 20a to the user through the sensor portions 160 and 260, respectively. Only the mobile fragrance discharge device 10 relatively close to the user may discharge a specific fragrance material. Also, when it is verified that the user desires to discharge a large amount of fragrance material based on psychological information of the user or a selection of the user, all of the mobile fragrance discharge device 10 and the external device 20a may operate to discharge the fragrance material. Although the mobile fragrance discharge device 10 is away from the user, the mobile fragrance discharge device 10 may move to a location close to the user by operating the propeller 190 and may discharge the fragrance material toward the user. When two or more users are present in the same space, each user may control a relatively close device between the mobile fragrance discharge device 10 and the external device 20a to discharge a desired fragrance material. Each of the mobile fragrance discharge device 10 and the external device 20a may verify a user relatively close thereto through the corresponding sensor portion 160 or 260 and may discharge a fragrance material most suitable for the corresponding user.

In addition, although the mobile fragrance discharge device 10 and the external device 20a are not present in the same space, the mobile fragrance discharge device 10 and the external device 20a may be disposed in mutually neighboring spaces based on a movement line of the user, or may be physically separate from each other and disposed in a temporally continuous space. In this case, the mobile fragrance discharge device 10 and the external device 20a may exchange mutual current states such that continuous emission may be provided to the user although the user moves.

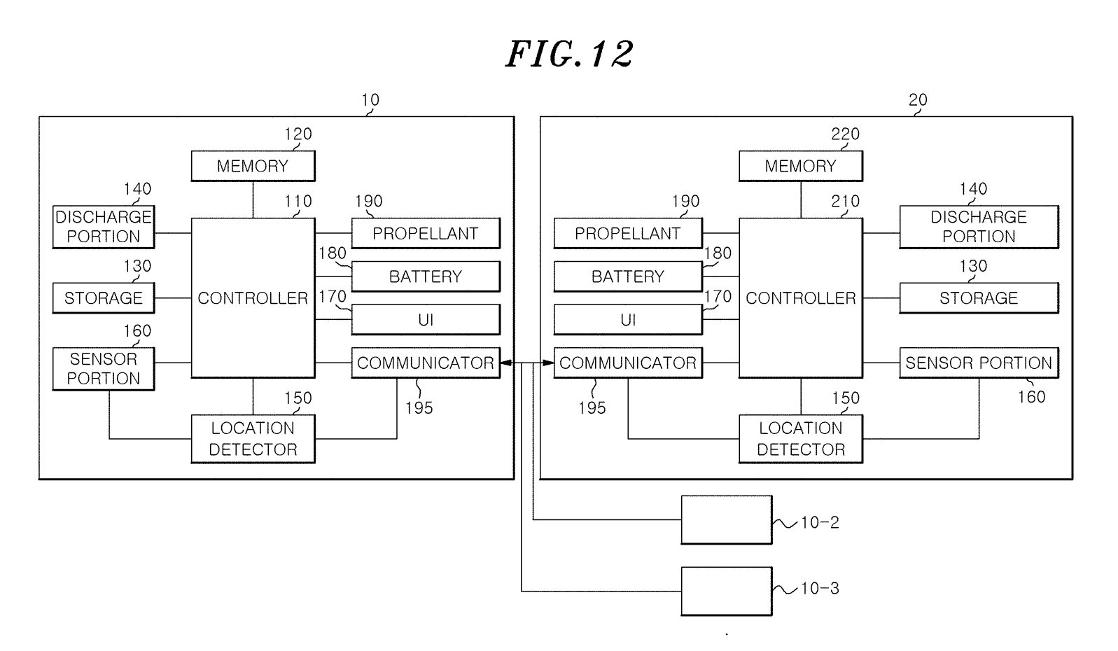

FIG. 12 is a block diagram illustrating another configuration of the mobile fragrance discharge device of FIG. 6 and another external device interacting therewith.

Referring to FIG. 12, each of external devices 10-1, 10-2, and 10-3 according to an embodiment refers to a device that may discharge a fragrance material and may be movable by including the propeller 190, and is configured to effectively perform discharging of the fragrance material within a predetermined space. Since each of the external devices 10-1, 10-2, and 10-3 has a configuration substantially identical to that of the mobile fragrance discharge device 10, a further description related thereto is omitted here. An interaction between the external device 10-1, 10-2, and 10-3 and the mobile fragrance discharge device 10 is further described.

The mobile fragrance discharge device 10 and the mobile fragrance discharge devices 10-1, 10-2, and 10-3 may interact with each other and each may be movable within the space by including the propeller 190. The mobile fragrance discharge device 10 and the mobile fragrance discharge devices 10-1, 10-2, and 10-3 may share at least one piece of information, such as their own location information within the space, travel route information, a relative distance from the user, and body information of the user through the communicator 195 and may interact with each other based on such information. For example, when the user moves within a predetermined space, the respective mobile fragrance discharge devices 10, 10-1, 10-2, 10-3 may share their locations through the communicator 195 so that any of the mobile fragrance discharge devices 10, 10-1, 10-2, 10-3 closest to the user may move toward the user and discharge a fragrance material. Also, when it is verified that the user desires to discharge a large amount of fragrance material based on psychological information of the user or a selection of the user, all of the mobile fragrance discharge devices 10, 10-1, 10-2, and 10-3 may operate to discharge the corresponding fragrance material.

Meanwhile, the mobile fragrance discharge device 10 may serve as a master and may control other mobile fragrance discharge devices 10-1, 10-2, and 10-3. The mobile fragrance discharge device 10 may determine a travel route of each of the mobile fragrance discharge devices 10, 10-1, 10-2, 10-3 based on location information, travel route information, environmental information, and user information detected through the other mobile fragrance discharge devices 10-1, 10-2, and 10-3. The determined travel route may be transmitted to the other mobile fragrance discharge devices 10-1, 10-2, and 10-3 through the communicator 195. Each of the other mobile fragrance discharge devices 10-1, 10-2, and 10-3 may operate the propeller 190 based on the received travel route and may control discharging of the fragrance within the travel route or at a specific location while moving.

### Control Method of Mobile Fragrance Discharge Device

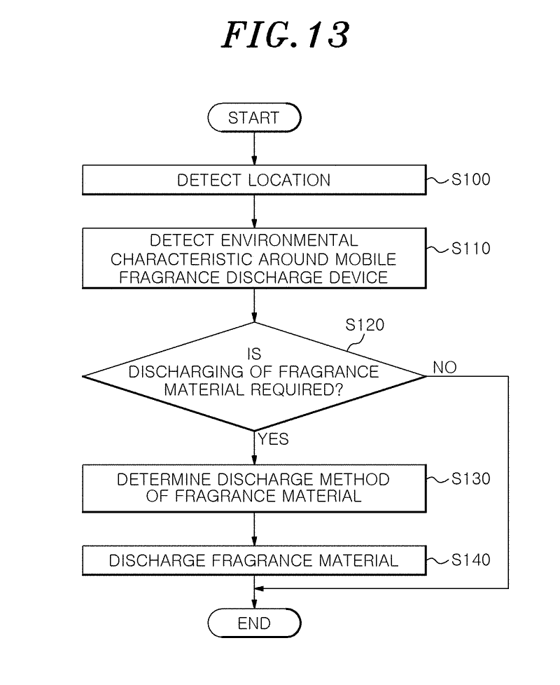

FIG. 13 is a flowchart illustrating a control method of a mobile fragrance discharge device according to an embodiment.

Referring to FIGS. 6 and 13, in operation S100, the mobile fragrance discharge device 10 detects its location through the location detector 150. The location information may be directly detected by the mobile fragrance discharge device 10 and may be received through the external device 20. In operation S110, the mobile fragrance discharge device 10 detects an environmental characteristic around the mobile fragrance discharge device 10 through the sensor portion 160. Here, the environmental characteristic around the mobile fragrance discharge device 10 may include environmental characteristic information detected at the sensor portion 160 or the sensor portion 260 of the external device 20 and environmental characteristic information received from the external device 20 of the user. The environmental characteristic may include at least one of a spatial characteristic of the space in which the mobile fragrance discharge device 10 is present, for example, a location and a size of the space, air pollution information, obstacle information, spatial information including a location of the user, time information, and odor information.

In operation S120, the mobile fragrance discharge device 10 determines whether discharging of the fragrance material is required based on the environmental characteristic and the location information or the travel route information. For example, when the user is cooking in a kitchen, the sensor portion 160 of the mobile fragrance discharge device 10 may determine whether cooking is ongoing using an odor sensor and may determine that there is a need to mask cooking smell in the corresponding space. Also, when the user uses a toilet, the sensor portion 160 may detect an unpleasant smell by use of the toilet and may determine there is a need to discharge the fragrance material in the toilet. Also, when the user is viewing multimedia through a multimedia content player, the sensor portion 160 may determine that there is a need to discharge the fragrance material around the user to increase a degree of immersion of the user. In operation S130, the controller 110 determines a discharge method of the fragrance material based on the verified environmental characteristic and the location information of the mobile fragrance discharge device 10. For example, the mobile fragrance discharge device 10 may move from a current location to the toilet or the kitchen and then may discharge the fragrance material. In this case, the mobile fragrance discharge device 10 may verify that the user has left the toilet or finished cooking and may move to the corresponding location and may discharge the fragrance material. Alternatively, the mobile fragrance discharge device 10 may move to the toilet or the kitchen in advance and wait there, and may discharge the fragrance material when the user finishes using the toilet or cooking.

Although discharging of the fragrance material is completed, the mobile fragrance discharge device 10 may continuously detect a change in the environmental characteristic. For example, the mobile fragrance discharge device 10 may continuously detect whether spatial characteristic information or user characteristic information changes, such as when the user leaves a room, when the user is to view multimedia using the multimedia play device, when cooking is ongoing within the space, or when the user is to take a rest or sleep. In this case, the mobile fragrance discharge device 10 may verify the spatial characteristic information and the user characteristic information using its sensor portion 160 and may also use user characteristic information or spatial characteristic information measured at the sensor portion 260 of the external device 20 in addition to the sensor portion 160. Also, the mobile fragrance discharge device 10 may use user information, for example, a daily schedule, a psychological state, and body information, stored by the external device 20. When a new environmental characteristic is detected in operation S110, the mobile fragrance discharge device 10 determines whether discharging of the fragrance material is required based on the verified new environmental characteristic and location information in operation S120. The mobile fragrance discharge device 10 determines the discharge method of fragrance material based on the new environmental characteristic or the location information, and the controller 110 controls the discharge portion 140 to discharge a new fragrance material based on the determined discharge method in operation S140.

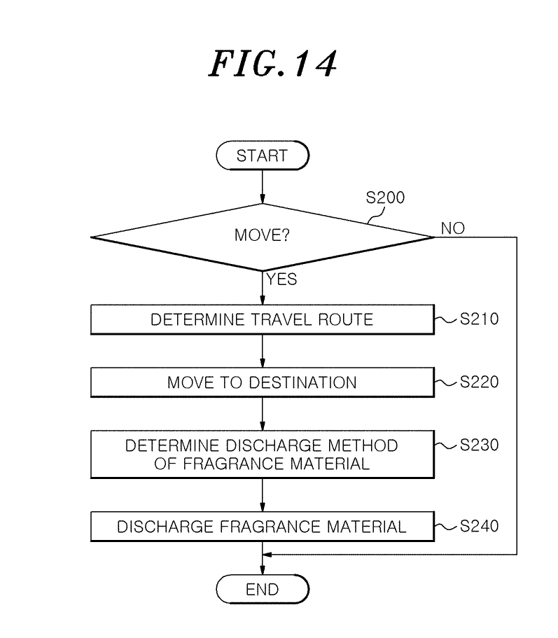

FIGS. 14 through 18 are flowcharts illustrating a control method of a mobile fragrance discharge device according to another embodiment.

Referring to FIGS. 6 and 14, in operation S200, the mobile fragrance discharge device 10 determines whether the mobile fragrance discharge device 10 needs to move. For example, the mobile fragrance discharge device 10 may connect to the external device 20 used by the user through the communicator 195. For example, the external device 20 may be a multimedia player. When the user desires to view movie through the external device 20, the external device 20 may call the mobile fragrance discharge device 10 to provide a fragrance material for providing the user with a sense of immersion. In this case, the external device 20 may transmit current location information of the user to the mobile fragrance discharge device 10 through the communicator 295. In response to receiving the location information of the user, the mobile fragrance discharge device 10 determines whether there is a need to move based on its location information detected at the location detector 150 in operation S200. When the mobile fragrance discharge device 10 is away from the user, the mobile fragrance discharge device 10 determines a travel route based on its location information, location information of the user, and spatial characteristic information in operation S210. When many persons are gathering within the travel route and an obstacle is present within the travel route, the mobile fragrance discharge device 10 may set the travel route to avoid the other persons and the obstacle in operation S210. In this case, the corresponding spatial information may be received from the external device 20 of the user or may be stored in advance in the memory 120 of the mobile fragrance discharge device 10. In operation S220, the mobile fragrance discharge device 10 operates the propeller 190 and moves to a destination. In this case, if an amount of the battery 180 is sufficient to quickly move to the destination, the mobile fragrance discharge device 10 may increase a travel speed by increasing a driving force of the propeller 190. In operation S230, the mobile fragrance discharge device 10 may arrive near to the destination and may determine a discharge method of the fragrance material. In this case, the mobile fragrance discharge device 10 may receive user information stored in the external device 20 of the user and may verify a preferred fragrance material of the user or body information of the user in operation S230 and accordingly, may determine the discharge method of the fragrance material in operation S240. For example, when the user information includes anomaly information of an olfactory organ (nasal cavity, oral cavity) of the user, the mobile fragrance discharge device 10 may select a fragrance material that does not stimulate the olfactory organ of the user based on the user information in operation S230 and may discharge the fragrance material in a direction in which the olfactory organ of the user is not stimulated.

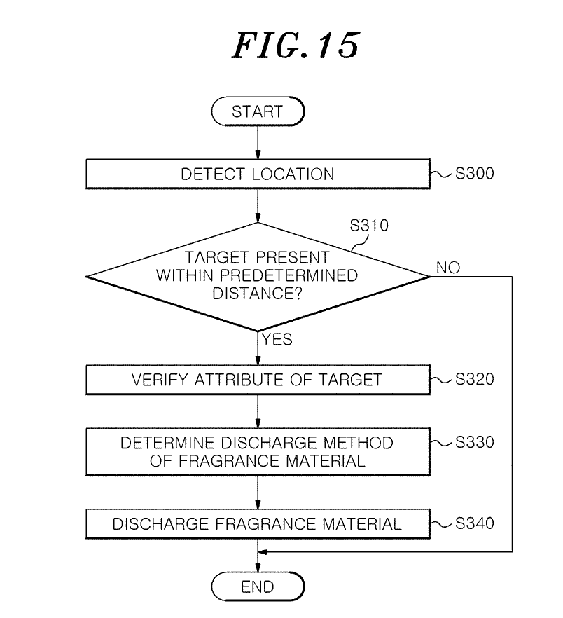

FIG. 15 is a flowchart illustrating a control method of the mobile fragrance discharge device 10 to discharge a fragrance material based on its location information and attribute information of a target.

Referring to FIGS. 6 and 15, in operation S300, the mobile fragrance discharge device 10 detects its location through the location detector. The location information may be directly detected by the mobile fragrance discharge device 10 and may be received through the external device 20. In operation S310, the mobile fragrance discharge device 10 verifies whether a target is present within a predetermined distance from the mobile fragrance discharge device 10 through the sensor portion 160. Here, the sensor portion 160 may include at least one of a proximity sensor, an ultrasonic sensor, an IR sensor, an odor sensor, an image sensor, and a camera. In operation S320, the controller 110 performs image processing based on information sensed through the sensor portion 160 and verifies an attribute of the target. In this case, the attribute of the target may be information on at least one of a type of the target, a size of the target, a color of the target, a material of the target, a movement of the target, and an odor. In operation S330, the controller 110 determines a discharge method of the fragrance material based on the verified attribute of the target and location information. Here, the discharge method of the fragrance material may determine at least one of a type of the fragrance material, an amount of the fragrance material, whether to discharge the fragrance material, a discharge direction, a discharge pattern, and a discharge location of the fragrance material relative to the target. For example, when a face of the user within a predetermined distance from the mobile fragrance discharge device 10 is recognized as the target, the discharge direction may be changed such that the fragrance material may not be directly discharged toward the face of the user. Also, when a plurality of persons is gathering within a space in which the mobile fragrance discharge device 10 is present, the mobile fragrance discharge device 10 may approach near to the target and discharge the fragrance material toward the target without obstructing other persons. Also, a type and intensity of the fragrance material may be changed depending on whether the target is a person or an animal. Also, when the target is the person, the fragrance material may be discharged by verifying a material of the target, for example, a material (leather, cloth) of clothes worn by the person or by adjusting a type, intensity, and/or a perfuming time of the fragrance material based on a size of the target. Also, the type of the fragrance material to be discharged may be changed depending on whether the target enters or leaves the space in which the mobile fragrance discharge device 10 is present. In operation S340, the controller 110 controls the discharge portion 140 to discharge the fragrance material based on the determined discharge method.

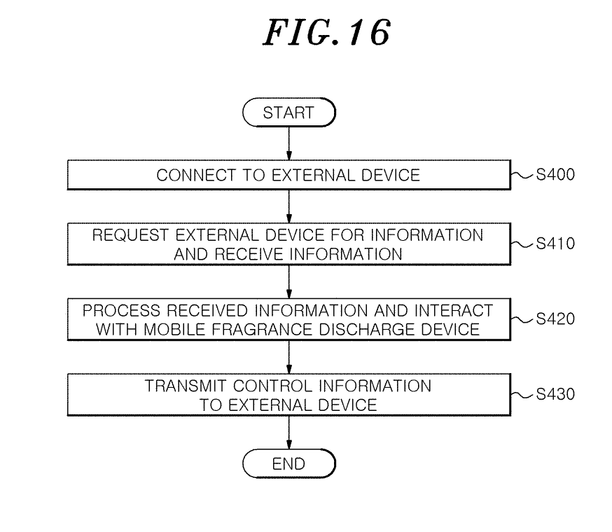

FIG. 16 is a flowchart illustrating an operation of actively controlling, by the mobile fragrance discharge device 10, the external device 20 in conjunction with the external device 20.

Referring to FIGS. 6 and 16, in operation S400, the mobile fragrance discharge device 10 connects to the external device 20 communicably connectable to the mobile fragrance discharge device 10. Here, the external device 20 refers to a device that may operate in conjunction with the mobile fragrance discharge device 10 and may be one of, for example, a window inside a room, an air conditioner, an air cleaner, a drying machine, a light, a ventilator, a humidifier, and a vacuum cleaner. The mobile fragrance discharge device 10 may connect to the external device 20 using IrDA, Bluetooth, ZigBee, or WiFi. Once the connection is completed, the mobile fragrance discharge device 10 requests the external device 20 for external device related information and receives the same in operation S410. In this case, the external device 20 may be, for example, a window that may be opened and closed automatically. If the mobile fragrance discharge device 10 is to ventilate the room by discharging a specific fragrance material in a space, the window may be closed in advance. When the mobile fragrance discharge device 10 attempts a communication connection with an actuator of the window and connects thereto, a current state (open/closed) of the window may be received and the mobile fragrance discharge device 10 may be activated based on the received state information in operation S420. That is, when the window is open, the mobile fragrance discharge device 10 may transmit control information for closing the window to the external device 20 in operation S430 and may transmit control information for opening the window to ventilate the room to the external device 20 before discharging the fragrance material in operation S430.

As another example, the external device 20 may be a light. When the user is to have a sleep in a room, the mobile fragrance discharge device 10 may discharge a fragrance material capable of inducing the user to have a good sleep. In this case, the mobile fragrance discharge device 10 may attempt a communication connection with the light and may receive current state information of the light so that the user may have a good sleep. Based on the received state information of the light, the mobile fragrance discharge device 10 may adjust a brightness of the light to be dark or may turn OFF the light the light is bright, and, conversely, may turn ON the light again after a preset period of time is elapsed. An action of the user to have a sleep may be verified from another device, such as a smartphone of the user interacting with the mobile fragrance discharge device 10. For example, the smartphone of the user capable of detecting a biorhythm of the user may detect the biorhythm, may verify the action of the user to have a sleep, and may transmit the corresponding information to the mobile fragrance discharge device 10. In this case, the mobile fragrance discharge device 10 may discharge a fragrance material suitable for the user to have a sleep based on the received information and, at the same time, may change an illuminance of the wirelessly connected light inside the room to be dark.

As another example, when an amount of a fragrance material stored in the storage 130 of the mobile fragrance discharge device 10 is insufficient, the mobile fragrance discharge device 10 may operate in conjunction with conditioning equipment, such as an air conditioner or a humidifier, to provide a further effect olfactory environment in a space in which the user is present. That is, the mobile fragrance discharge device 10 attempts a communication connection with an air conditioner or a humidifier that is the external device 20 in operation S400. Once the connection is completed, the mobile fragrance discharge device 10 requests and receives air conditioner or humidifier related information in operation S410. The mobile fragrance discharge device 10 may operate based on the received state information of the air conditioner or the humidifier in operation S420, and may control the air conditioner or the humidifier such that the fragrance material may be further effectively diffused in the space in operation S430.

As another example, the external device 20 may be a multimedia player such as a television (TV), an audio system, and a headset. The mobile fragrance discharge device 10 may play the multimedia player to provide a further effective olfactory environment while discharging a fragrance material.

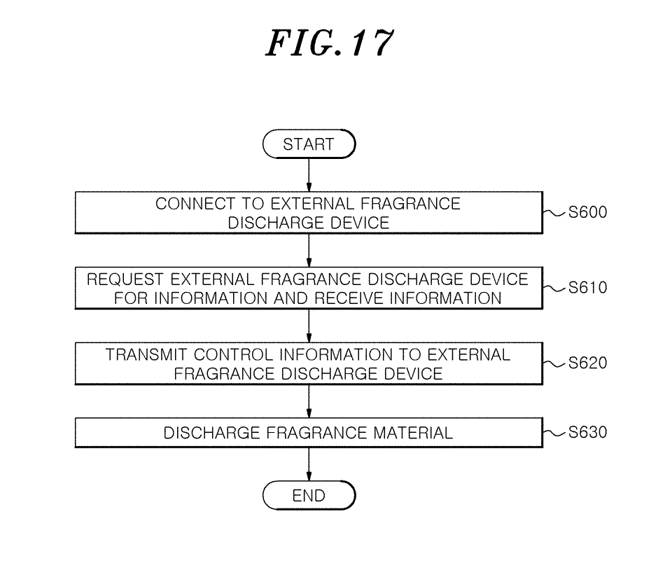

FIG. 17 is a flowchart illustrating an operation of performing, by the mobile fragrance discharge device 10, a fragrance material discharge in conjunction with an external device capable of discharging a fragrance material.

Referring to FIGS. 12 and 17, in operation S600, the mobile fragrance discharge device 10 connects to the external devices 10-1, 10-2, and 10-3 each communicably connectable to the mobile fragrance discharge device 10 and capable of discharging a fragrance material. Here, the external device 10-1, 10-2, and 10-3 refers to a device that may discharge the fragrance material and may be a product functionally similar to the mobile fragrance discharge device 10. The mobile fragrance discharge device 10 may connect to the external device using IrDA, Bluetooth, ZigBee, or WiFi. Once the connection is completed, the mobile fragrance discharge device 10 requests the external device for external device related information and receives the same in operation S610. In this case, information associated with the external device may include at least one of a location of the external device, a type and an amount of the fragrance material stored in a storage of the external device, user information sensed at the sensor portion of the external device, and environmental information. In operation S620, the mobile fragrance discharge device 10 may determine a discharge method of the fragrance material within the space based on at least one of the user information sensed at the sensor portion 160, the environmental information, and type and amount information of the fragrance material stored in the storage 130 and may transmit control information associated with fragrance material discharge of the external device. In operation S630, the mobile fragrance discharge device 10 may control the discharge portion 140 of the mobile fragrance discharge device 10 to discharge the fragrance material. The external device controls the discharge portion to discharge the fragrance material based on the control information received from the mobile fragrance discharge device 10.

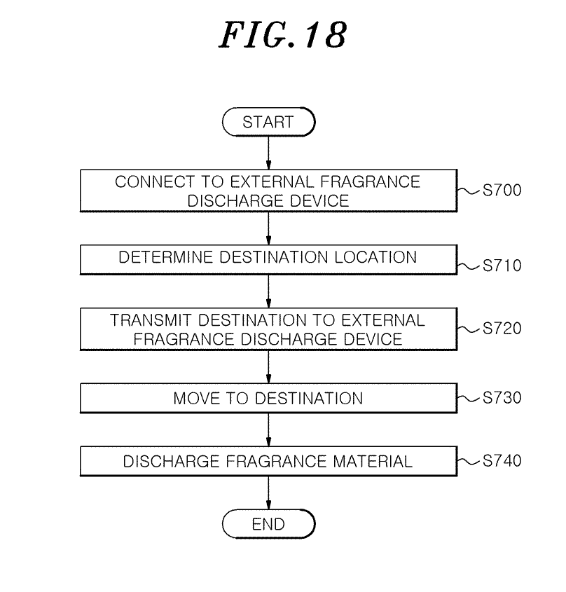

FIG. 18 is a flowchart illustrating another operation of performing, by the mobile fragrance discharge device 10, a fragrance material discharge in conjunction with an external device capable of discharging a fragrance material.

Referring to FIGS. 12 and 18, in operation S700, the mobile fragrance discharge device 10 connects to the external devices 10-1, 10-2, and 10-3 each communicably connectable to the mobile fragrance discharge device 10 and capable of discharging a fragrance material. Here, the external device refers to a device that may discharge the fragrance material and may be a product substantially similar to the mobile fragrance discharge device 10. The mobile fragrance discharge device 10 may connect to the external device using IrDA, Bluetooth, ZigBee, or WiFi. Once the connection is completed, the mobile fragrance discharge device 10 requests the external device for external device related information and receives the information. The external device related information may include current location information of the external device, battery information, information on a type and a stored amount of the fragrance material. The mobile fragrance discharge device 10 determines a destination location at which the fragrance material is to be discharged in operation S710 and transmits destination location information to the external device in operation S720. Here, when a plurality of external devices is provided and each of the external devices is away from the destination, the mobile fragrance discharge device 10 may determine a discharge method of a fragrance material for providing optimal effect to the user present at the destination based on battery information and information on a type and a stored amount of a fragrance material of each of the external devices. For example, the mobile fragrance discharge device 10 may select an external device of which a remaining battery amount is sufficient and that stores a fragrance material suitable for an emotional state of the user, and may transmit destination information only to the selected external device in operation S720. In the meantime, the mobile fragrance discharge device 10 may directly determine a travel route of each of the external devices 10-1, 10-2, and 10-3 based on a location of each of the external devices, spatial information and a location of the destination and may transmit the travel route to each of the external devices. Each of the external devices moves to the destination based on the received destination location or travel route in operation S730 and discharges a suitable fragrance material in operation S740.

### Example of Using Mobile Fragrance Discharge Device

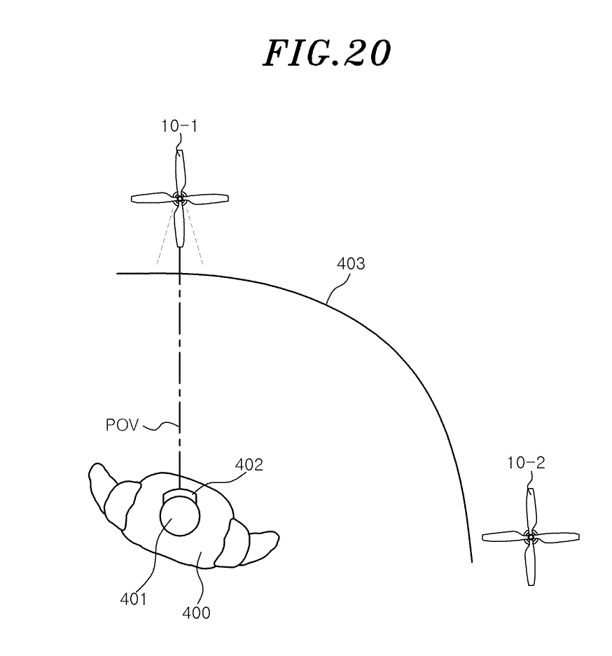

FIGS. 19 and 20 illustrate examples in which a mobile fragrance discharge device interacts with a multimedia player according to an embodiment.

Referring to FIGS. 19 and 20, a user 400 is wearing an HMD 402 around a head 401. The mobile fragrance discharge devices 10-1 and 10-2 are disposed on the left and the right of the user 400, respectively. Each of the mobile fragrance discharge devices 10-1 and 10-2 includes a configuration of the mobile fragrance discharge device 10 of FIG. 6. Each of the mobile fragrance discharge devices 10-1 and 10-2 may communicably connect to the HMD 402. The HMD 402 includes a configuration of the external device 20 of FIG. 6. The user 400 may experience virtual reality through the HMD 402. A screen 403 refers to a virtual reality image provided to the user 400 through the HMD 402 worn by the user 400 and tracks a point of view (POV) of the user 400 and displays a screen corresponding to the POV in response to rotating the head 401 of the user 400. Referring to FIG. 12A, if the user 400 turns the head 401 to the left, the POV of the user 400 faces a flowerbed displayed on the left of the screen 403. In this case, the mobile fragrance discharge device 10-1 close to the POV of the user 400 may operate by interacting with the HMD 402. The HMD 402 may analyze an image of the flowerbed currently present on the POV of the user 400 and may transmit corresponding information to the mobile fragrance discharge device 10-1. The mobile fragrance discharge device 10-1 may discharge a suitable fragrance material, for example, a rose fragrance, toward the user 400 based on image information received from the HMD 402. That is, the mobile fragrance discharge device 10-1 may determine olfactory environmental information associated with an image corresponding to virtual location information on a virtual reality space on which the POV of the user 400 is present, which varies in response to a movement of the head 401 of the user 400, and may discharge the suitable fragrance material. In this case, the mobile fragrance discharge device 10-1 may analyze information of the user 400 sensed at the sensor portion 160 of the mobile fragrance discharge device 10-1, may verify a location of an olfactory organ (lips, nose) of the user 400, and may dispense the fragrance material toward the verified olfactory organ at appropriate intensity.

In the meantime, if the POV of the user 400 directs the right, that is, a tree on the screen 403, the mobile fragrance discharge device 10-2 closest to the POV of the user 400 may operate by interacting with the HMD 402. The mobile fragrance discharge device 10-2 may discharge a suitable fragrance material, for example, a fragrance material emitting a forest fragrance, toward the user 400 based on image information received from the HMD 402. Also, the mobile fragrance discharge devices 10-1 and 10-2 may simultaneously discharge suitable fragrance materials based on the overall atmosphere of an image displayed on the HMD 402, regardless of the POV of the user 400. When the user 400 is into a game situation without moving during a preset period of time or more in a game space using the HMD 402, it is possible to increase a degree of immersion of the user 400 into the game by discharging a fragrance material suitable for an image of a corresponding section displayed on the HMD 402.

Also, a variety of sensors, for example, an electrocardiogram (ECG) sensor, an electroencephalogram (EEG) sensor, a photo-plethysmography (PPG) sensor, a blood oxygen saturation level (S_{P}O₂) sensor, a brainwave sensor, and an image sensor, may be mounted to the HMD 402. The HMD 402 may measure physical condition information of the user 400 using the sensor, may verify an emotion or a feeling of the user 400 based on the measured physical condition information, and may transmit corresponding information to the mobile fragrance discharge devices 10-1 and 10-2 to discharge a fragrance material suitable for the current emotion or feeling of the user 400.

The mobile fragrance discharge devices 10-1 and 10-2 may move with maintaining a predetermined distance from the user 400 while tracking the user 400 in accordance with a motion of the user 400. That is, when the user 400 moves, each of the mobile fragrance discharge devices 10-1 and 10-2 may move along a travel route of the user 400 with maintaining a relative distance from the user 400 while sharing mutual locations. Also, each of the mobile fragrance discharge devices 10-1 and 10-2 may verify a location of a respiratory organ (oral cavity or nasal cavity) of the user 400 and may target a nozzle through which a fragrance material is discharged around the respiratory organ of the user 400 at all times while flying around the verified location. Accordingly, a fragrance material may be provided immediately at a point in time at which the fragrance material needs to be provided to the user 400, which may lead to increasing a user satisfaction.

Also, each of the mobile fragrance discharge devices 10-1 and 10-2 may track a movement of a head 401 of the user 400 through the sensor portion 160. For example, each of the mobile fragrance discharge devices 10-1 and 10-2 may sense an action of the user 400 and a movement of the head 401 of the user 400 through an image sensor of the sensor portion 160. If the movement of the user 400 varies by a preset value or more, each of the mobile fragrance discharge devices 10-1 and 10-2 may move to a location corresponding to the target of which the movement is changed. If the movement of the user 400 is insignificant and only a direction of a nozzle through which the fragrance material is discharged needs to be switched, each of the mobile fragrance discharge devices 10-1 and 10-2 may switch only the direction of the nozzle while maintaining a current location. Each of the mobile fragrance discharge devices 10-1 and 10-2 may also receive movement information of the head 401 of the user 400 through a motion sensor, such as an acceleration sensor mounted to the HMD 402.

Also, to generate an olfactory environment suitable for a video played at a current point in time and a future point in time of VR video information provided on the HMD 402, each of the mobile fragrance discharge devices 10-1 and 10-2 may determine in advance a point in time at which a fragrance material for the corresponding video is to be discharged and may move to a corresponding location. In this case, to increase concentration of fragrance, the mobile fragrance discharge devices 10-1 and 10-2 may move in a near distance from the user 400, an amount of a fragrance material to be discharged may increase, and the diffusing power of the fragrance material may be enhanced by mounting a fan (not shown) to the mobile fragrance discharge device 10-1, 10-2 and by rotating the fan.

The HMD 402 may track a POV of the user 400 and may transmit information of a target present on a VR video corresponding to the POV to the mobile fragrance discharge devices 10-1 and 10-2. Each of the mobile fragrance discharge devices 10-1 and 10-2 may discharge a suitable fragrance material based on the received target information. For example, if the POV of the user 400 directs flowers on the VR video, each of the mobile fragrance discharge devices 10-1 and 10-2 may discharge a fragrance of flowers while moving toward the user 400. If the POV of the user directs fruits on the VR video, each of the mobile fragrance discharge devices 10-1 and 10-2 may discharge a fragrance of fruits.

Meantime, each of the mobile fragrance discharge device 10-1, 10-2 may receive information on a psychological state or a health state of the user 400 in conjunction with a mobile device such as a smartphone of the user 400. Each of the mobile fragrance discharge device 10-1, 10-2 may select a suitable fragrance material based on the received information on the psychological state or the health state and move to be around the user 400 and then may discharge the selected fragrance material. At the same time, each of the mobile fragrance discharge device 10-1, 10-2 may transmit information on the psychological state or the health state of the user 400 to the multimedia player such as the HMD 402 so that a video suitable for the psychological state or the health state of the user 400 may be played on the multimedia player.

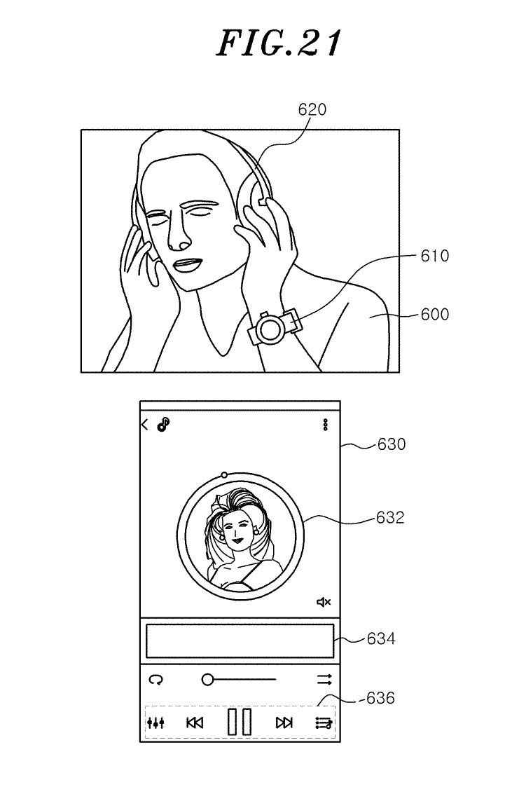

FIG. 21 illustrates an example in which a mobile fragrance discharge device interacts with another multimedia player according to an embodiment.

Referring to FIG. 21, a user 600 is wearing a headset 620 around a head and also wearing a smartwatch 610 around a wrist. A variety of sensors, for example, an ECG sensor, an EEG sensor, a PPG sensor, an S_{P}O₂ sensor, a brainwave sensor, and an image sensor, may be mounted to the headset 620. Also, a variety of sensors capable of measuring ECG or a psychological state of the user 600 may be mounted to the smartwatch 610. The user 600 may listen to music playing on a multimedia player 630 of the user 600 through the headset 620. The multimedia player 630 may include jacket information 632 on a currently playing album, lyrics information 634 on lyrics of a currently playing song, and control information 636 used to select music to be played and control sound effect. Although not illustrated, a mobile fragrance discharge device may be provided around the user 600. The mobile fragrance discharge device may include a configuration of the mobile fragrance discharge device 10 of FIG. 6 and the multimedia player 630 may include a configuration of the external device 20 of FIG. 6.

To enhance a degree of immersion on music of the user 600 listening to the music playing on the multimedia player 630 through the headset 620, the multimedia player 630 may transmit information associated with the playing music, for example, the jacket information 632, a music bit, a music style, and the lyrics information 634 to the mobile fragrance discharge device. The mobile fragrance discharge device may select and discharge a fragrance material capable of enhancing the degree of immersion of the user 600 based on the received music related information. Through the variety of sensors mounted to the headset 620 and the variety of sensors mounted to the smartwatch 610, information associated with a psychological state and a health state of the user 600 may be verified. Additionally, information associated with the psychological state and the health state of the user 600 may be verified based on social media activity information (Facebook, Twitter) and schedule information of the user 600. The headset 620 or the smartwatch 610 may transmit, to the mobile fragrance discharge device, the sensed location information, health state, or psychological state information. The mobile fragrance discharge device may discharge a suitable fragrance material based on the received user state information and location information and location information of the mobile fragrance discharge device. If a plurality of users is listening to the same music within the same space, the mobile fragrance discharge device may select and discharge a suitable fragrance material based on average heath state or psychological state information of the plurality of users present within the space.

If music playing on the multimedia player 630 changes, the mobile fragrance discharge device may appropriately change and discharge a type, a discharge direction, intensity, etc., of a fragrance material to be discharged. When the mobile fragrance discharge device does not store an appropriate fragrance material, the mobile fragrance discharge device may call another mobile fragrance discharge device and then move to a place for battery charging. Also, the multimedia player 630 may measure the psychological state of the user 600 with respect to the fragrance material that is discharged from the mobile fragrance discharge device using the sensors mounted to the headset 620 or the sensors mounted to the smartwatch 610, and may receive psychological state information of the user 600 that is verified based on the measured physical condition information of the user 600 and may store the same as olfactory environmental information of the user 600. Accordingly, if the same user 600 subsequently listens to the same music again through the multimedia player 630, the mobile fragrance discharge device may discharge the previous fragrance material based on the stored olfactory environmental information of the user 600 and may provide the user 600 with the degree of immersion the user 600 has previously perceived.

Although it is described herein as an example that the multimedia player 630 is a music player, the same effect may be achieved when the multimedia player 630 is a smartphone of the user, a TV, a PDA, a laptop computer, a PC, or a digital camera. For example, the user may wear the smartwatch 610 capable of measuring the psychological state or the health state of the user 600 and verifying an emotion or a feeling of the user 600 based on the measured physical condition information and, in this state, may capture an image of an object using a digital camera capable of interacting with the smartwatch 610. In this case, the smartwatch 610 may verify and store a health state and a psychological state of the user 600 at a capturing point in time or an atmosphere around a place where capturing is performed. Subsequently, when the user 600 views the corresponding image using the multimedia player 630, the mobile fragrance discharge device interacting with the multimedia player 630 may move to be around the user 600 and may determine and discharge a fragrance material based on the stored health state or psychological state of the user 600 or atmosphere information around the space, so that the user 600 may be immersed into the atmosphere at the time of capturing.

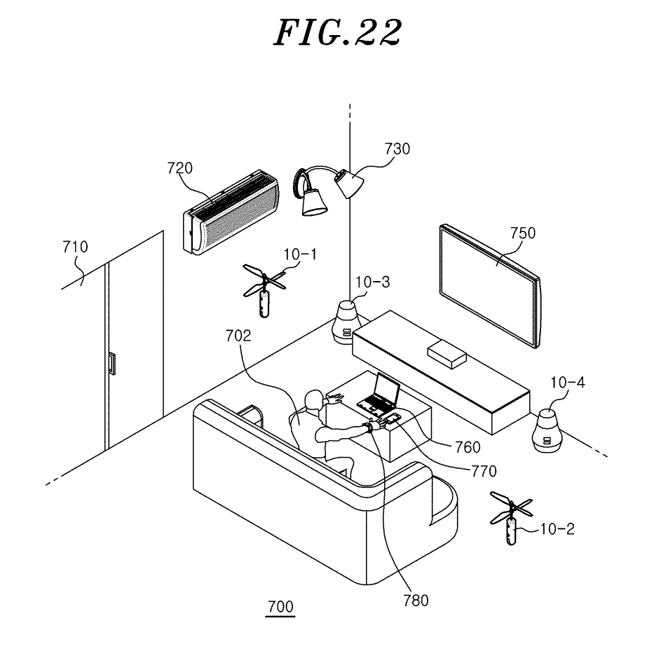

FIG. 22 illustrates an example in which a mobile fragrance discharge device interacts with various external devices according to an embodiment.

Referring to FIG. 22, a plurality of mobile fragrance discharge devices 10-1 and 10-2 and various external devices 710, 720, 730, 750, 760, 770, and 780 and stationary fragrance discharge devices 10-3 and 10-4 capable of interacting with the mobile fragrance discharge devices 10-1 and 10-2 are present within a space 700 in which a user 702 is present. Each of the mobile fragrance discharge devices 10-1 and 10-2 may include a configuration of the mobile fragrance discharge device 10 of FIG. 6, and each of the external devices 710, 720, 730, 750, 760, 770, and 780 may include a configuration of the external device 20 of FIG. 6. The stationary fragrance discharge devices 10-3 and 10-4 include the same configuration as that of the mobile fragrance discharge devices 10-1 and 10-2 except for a propeller and a location detector. The mobile fragrance discharge devices 10-1 and 10-2 and the external devices 710, 720, 730, 750, 760, 770, and 780 and the stationary fragrance discharge devices 10-3 and 10-4 may connect to each other to be directly communicable using WiFi, ZigBee, IrDA, Bluetooth communication, and the like. For example, the mobile fragrance discharge device 10-1 may function as a master to control the other mobile fragrance discharge device 10-2 and the external devices 710, 720, 730, 750, 760, 770, and 780 and the stationary fragrance discharge devices 10-3 and 10-4. Also, the mobile fragrance discharge devices 10-1 and 10-2 and the external devices 710, 720, 730, 750, 760, 770, and 780 and the stationary fragrance discharge devices 10-3 and 10-4 may communicably connect to each other over an Internet network using an access point (not shown).

The mobile fragrance discharge device 10-1 may control discharging of a fragrance material within the space 700 based on characteristic information of the space 700 sensed using the other mobile fragrance discharge device 10-2 and the various external devices 710, 720, 730, 750, 760, 770, and 780, characteristic information of the user 702, and its location information. For example, the mobile fragrance discharge device 10-1 may detect an open/closed state of the window 710 and may control opening/closing of the window 710, and may control diffusion of a fragrance material in conjunction with the air conditioner 720. Also, the mobile fragrance discharge device 10-1 may verify the atmosphere of the space 700 based on a lighting state of the light 730 and may discharge a suitable fragrance material based on the verified atmosphere of the space 700 in conjunction with the other mobile fragrance discharge device 10-2. Each of the mobile fragrance discharge devices 10-1 and 10-2 may discharge a suitable fragrance material while tracking the user 702. In this case, each of the mobile fragrance discharge devices 10-1 and 10-2 may control a suitable fragrance material to be discharged based on a current location while tracking the user 702. For example, if a distance from the user 702 increases, each of the mobile fragrance discharge devices 10-1 and 10-2 may increase an amount of a fragrance material. If the user 702 turns the user's head, a mobile fragrance discharge device present at a location corresponding to an olfactory organ of the user may discharge a fragrance material. The mobile fragrance discharge devices 10-1 and 10-2 may receive multimedia content information playing on the television 750, the laptop PC 760, and the smartphone 770, and may receive psychological state information of the user 702 through various types of biometric sensors mounted to the smartwatch 780 worn by the user 702. The mobile fragrance discharge devices 10-1 and 10-2 may discharge a fragrance material based on the received information so that the user 702 may be further immersed into multimedia contents.

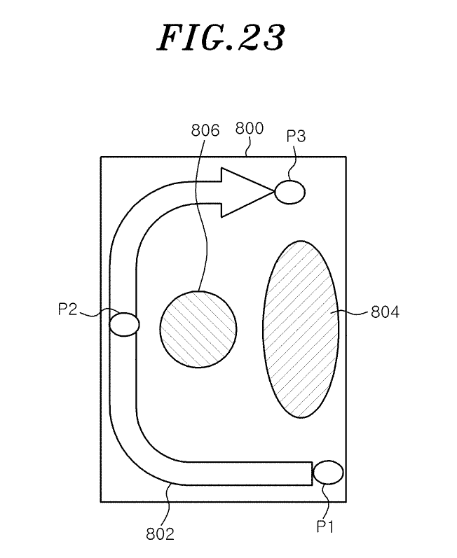

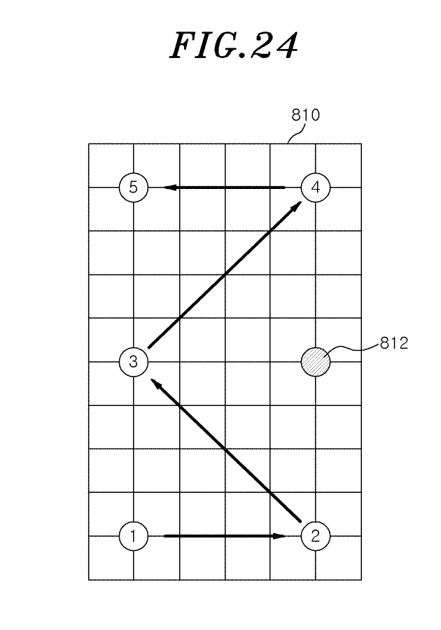

FIG. 23 illustrates an example of setting a path of a mobile fragrance discharge device according to an embodiment, and FIG. 24 illustrates another example of setting a path of a mobile fragrance discharge device according to an embodiment.

Referring to FIG. 23, the mobile fragrance discharge device is present within a space 800. Initially, the mobile fragrance discharge device is present at a location P1 within the space 800. A location detector of the mobile fragrance discharge device may determine P3 as a final destination and may determine a travel route for moving to P3. In this case, the mobile fragrance discharge device may receive environmental information around the space 800 from an external device or may use environmental information around the space 800 pre-stored in a memory of the mobile fragrance discharge device. Also, the mobile fragrance discharge device may sense and verify the environmental information through its sensor portion. For example, the mobile fragrance discharge device may verify that an obstacle 806 is present within the space 800 and that cooking is ongoing in a kitchen 804. The mobile fragrance discharge device may determine the travel route within the space 800 based on the verified information. For example, since the obstacle 806 is present within the space 800 and cooking is currently ongoing in the kitchen 804, the mobile fragrance discharge device may detect a travel route 802 that enables the mobile fragrance discharge device to make a detour around the obstacle 806 and to maximally cover the space 800. That is, the mobile fragrance discharge device may detect the travel route 802 starting from P1, passing through P2, and arriving at P3. Also, the mobile fragrance discharge device may determine a discharge characteristic of the fragrance material based on location information or travel route information. For example, to prevent taste of food from being degraded due to discharging of the fragrance material, the mobile fragrance discharge device may not discharge the fragrance material around the kitchen 804, for example, P1 in which cooking is ongoing. When the cooking is completed, the mobile fragrance discharge device may discharge the fragrance material to remove a remaining smell of food. To remove an unpleasant feeling by the smell of food in a space away from the kitchen 804, the mobile fragrance discharge device may discharge the fragrance material while moving along the corresponding route, for example, from P1 to P2 and from P2 to P3. Also, once the mobile fragrance discharge device arrives at the destination P3, the mobile fragrance discharge device may stop discharging of the fragrance material to prevent the fragrance material from being discharged toward the kitchen 804.

Referring to FIG. 24, the mobile fragrance discharge device is present within a space 810. A plurality of persons 812 is gathering in a middle portion on the right of the space 810. The mobile fragrance discharge device is present at a first location within the space 810. Here, currently, although an amount of a specific fragrance material stored in the mobile fragrance discharge device is insufficient, the mobile fragrance discharge device needs to quickly provide a fragrance within the space 810 with the specific fragrance material. In this case, the mobile fragrance discharge device needs to quickly discharge the fragrance material without obstructing the persons 812 and with maximally covering an inside of the space 810. Accordingly, the location detector of the mobile fragrance discharge device may detect a zig-zagged travel route from the first location to a fifth location.

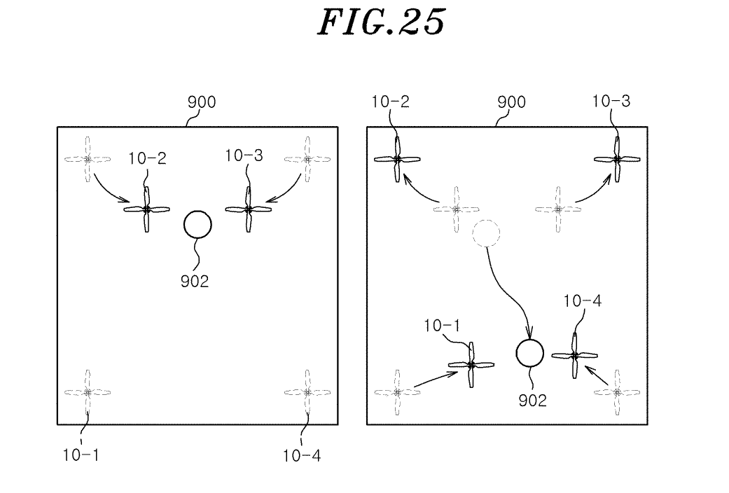

FIG. 25 illustrates an example in which a plurality of mobile fragrance discharge devices operates through mutual interaction according to an embodiment.

Referring to FIG. 25, four mobile fragrance discharge devices 10-1, 10-2, 10-3, and 10-4 are present at corners within a space 900, respectively. The four mobile fragrance discharge devices 10-1, 10-2, 10-3, and 10-4 may communicably connect to each other. Also, each of the four fragrance discharge devices 10-1, 10-2, 10-3, and 10-4 may adjust a direction of a nozzle in which a fragrance material is discharged in any direction. Each of the mobile fragrance discharge devices 10-1, 10-2, 10-3, and 10-4 may include a configuration of the mobile fragrance discharge device 10 of FIG. 6. Each of the mobile fragrance discharge devices 10-1, 10-2, 10-3, and 10-4 may verify environmental information around the space 900, such as a size, a shape, a pollution level of the space 900, and user information, such as a relative location of a target, that is, a user 902 and clothes and a size of the user 902 within the space 900 using its sensor portion 160, for example, an IR sensor, a laser sensor, an odor sensor, a proximity sensor, an image sensor, an ultrasonic sensor, a Bluetooth Low Energy (BLE) sensor, an air pollution measurement sensor, a temperature sensor, an illuminance sensor, and the like. Each of the mobile fragrance discharge devices 10-1, 10-2, 10-3, and 10-4 may transmit the verified information to another mobile fragrance discharge device and the other mobile fragrance discharge device may control discharging of a fragrance material based on the received information.

Referring to a figure on the left of FIG. 25, when the user 902 initially starts to play a multimedia player such as an HMD, the mobile fragrance discharge device 10-2 or 10-3 closest to the multimedia player or to the user 902 may move around the user 902 in conjunction with the multimedia player. While moving, the corresponding mobile fragrance discharge device 10-2 or 10-3 may discharge a fragrance material toward the user 902 to provide a fragrance material suitable for an emotional state or a psychological state of the user 902 or content information played on the multimedia player. When the mobile fragrance discharge device arrives around the user 902, the mobile fragrance discharge device may fly around the user 902 with tracking a movement of the user 902 and maintaining a predetermined distance from the user 902. In this case, each of the mobile fragrance discharge devices 10-2 and 10-3 may determine a suitable fragrance material based on content information of a specific section being played on the multimedia device or a change in an emotional state or a psychological state of the user 902 and may discharge the fragrance material while flying in accordance to a change in a location of an olfactory organ corresponding to the movement of the user 902. Each of the mobile fragrance discharge devices 10-1 and 10-4 relatively away from the user 902 may not discharge a fragrance material and may dock to a charging device (not shown) and may charge a battery.

Referring to a figure on the right of FIG. 25, if the user 902 further moves down within the space 900, the mobile fragrance discharge devices 10-1 and 10-4 may be located closer to the user 902. In this case, the mobile fragrance discharge devices 10-2 and 10-3 may return to original locations at which their own charging devices are present. At the same time, each of the mobile fragrance discharge devices 10-1 and 10-4 starts to move toward the user 902 with discharging a suitable fragrance material toward the user 902. When the mobile fragrance discharge devices 10-1 and 10-4 arrive around the user 902, the mobile fragrance discharge devices 10-1 and 10-4 may move around the user 902 with tracking a motion of the user 902 and maintaining a predetermined distance from the user 902, and at the same time, may discharge an appropriate fragrance material toward the user 902. As described above, the mobile fragrance discharge devices 10-1, 10-2, 10-3, and 10-4 disposed at predetermined locations within the space 900 may interact with each other and may verify and share the environmental information around the space 900 and the user information. Accordingly, it is possible to further effectively discharge a fragrance material within the space 900.

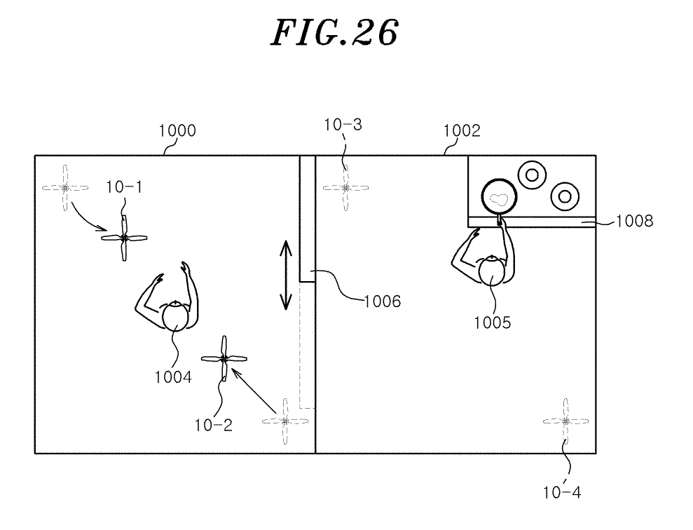

FIG. 26 illustrates another example in which a plurality of mobile fragrance discharge devices operates through mutual interaction according to an embodiment.

Referring to FIG. 26, two spaces, for example, a first space 1000 and a second space 1002, are adjacent to each other and are separate by a door 1006. The two spaces 1000 and 1002 may communicate with each other or may be blocked through sliding of the door 1006. Herein, description is made based on an example in which the first space 1000 is a living room and the second space 1002 is a kitchen. Two mobile fragrance discharge devices 10-1 and 10-2 are disposed at both corners to diagonally face each other within the first space 1000 and two mobile fragrance discharge devices 10-3 and 10-4 are disposed at both corners to diagonally face each other within the second space 1002. The mobile fragrance discharge devices 10-1, 10-2, 10-3 and 10-4 may communicably connect to each other. Also, the door 1006 may communicably connect to each of the mobile fragrance discharge devices 10-1, 10-2, 10-3, and 10-4 and may be automatically opened and closed by an actuator (not shown). Also, each of the mobile fragrance discharge devices 10-1, 10-2, 10-3 and 10-4 may adjust a direction of a nozzle in which a fragrance material is discharged in any direction. Each of the mobile fragrance discharge devices 10-1, 10-2, 10-3 and 10-4 may include a configuration of the mobile fragrance discharge device 10 of FIG. 6. Accordingly, each of the mobile fragrance discharge devices 10-1, 10-2, 10-3 and 10-4 may verify environmental information around the corresponding space 1000 or 1002, such as a size and a shape of the space 1000 or 1002, and user information, such as a relative location of a corresponding target, that is, a user 1004 or 1005 clothes and a size of the user 1004 or 1005 within the space 1000 or 1002 using its sensor portion 160.

The user 1004 within the first space 1000 is taking a rest and the user 1005 within the second space 1002 is cooking using a gas stove 1008. At an initial stage, the door 1006 is open to open the first space 1000 and the second space 1002. Once the user 1005 within the second space 1002 starts cooking, the mobile fragrance discharge devices 10-3 and 10-4 disposed within the second space 1002 may verify that the cooking is ongoing within the second space 1002. In detail, each of the mobile fragrance discharge devices 10-3 and 10-4 may verify that the cooking is ongoing within the second space 1002 using the odor sensor of its sensor portion 160. Alternatively, each of the mobile fragrance discharge devices 10-3 and 10-4 may communicably connect to the gas stove 1008 and verify that the cooking is ongoing if the gas stove 1008 is ON during a preset period of time or more. If the user 1005 within the second space 1002 starts cooking, a smell of cooking may be spread into the first space 1000 and may cause the user 1004 taking a rest within the first space 1000 to feel unpleasant. To prevent this, when the mobile fragrance discharge devices 10-3 and 10-4 detect that the cooking is ongoing within the second space 1002, the mobile fragrance discharge devices 10-3 and 10-4 may transmit corresponding information to the mobile fragrance discharge devices 10-1 and 10-2 within the first space 1000 and may operate the door 1006 to block the first space 1000 and the second space 1002 from each other. Each of the mobile fragrance discharge devices 10-1 and 10-2 within the first space 1000 may move toward the user 1004 and may discharge a suitable fragrance material around the user 1004 so that the user 1004 may take a further comfortable rest. Also, when the cooking is completed within the second space 1002, each of the mobile fragrance discharge devices 10-3 and 10-4 may discharge a suitable fragrance material toward the second space 1002 to remove a remaining smell of food and to refresh the second space 1002 to be atmospherically pleasant. Also, when the cooking is not ongoing within the second space 1002 and the door 1006 is opened such that the first space 1000 and the second space 1002 are opened to each other, the four mobile fragrance discharge devices 10-1, 10-2, 10-3, and 10-4 may interact with each other and may discharge a suitable fragrance material over the entire first space 1000 and second space 1002.

As described above, a mobile fragrance discharge device according to embodiments may determine a discharge characteristic of a fragrance material based on location information or travel route information and may control discharging of the fragrance material based on the determined discharge characteristic of the fragrance material.

The aforementioned description is provided as an example only. The mobile fragrance discharge device according to embodiments may be applicable to other various fields. For example, the mobile fragrance discharge device may verify an emotional state or a psychological state of a user appreciating a work of art based on social media activity information or a sensor mounted to a wearable device or a smartphone of the user. The smartphone or the wearable device may transmit corresponding information to the mobile fragrance discharge device. The mobile fragrance discharge device may provide a fragrance material suitable for the user based on the corresponding information. When the user appreciates the corresponding work of art again, the mobile fragrance discharge device may discharge a suitable fragrance material based on user information stored in a memory of the mobile fragrance discharge device.

Also, the mobile fragrance discharge device according to an embodiment may include a voice recognizer capable of recognizing voice of the user, and may analyze the voice in response to a call from the user, may identify the user, and may move toward the user.

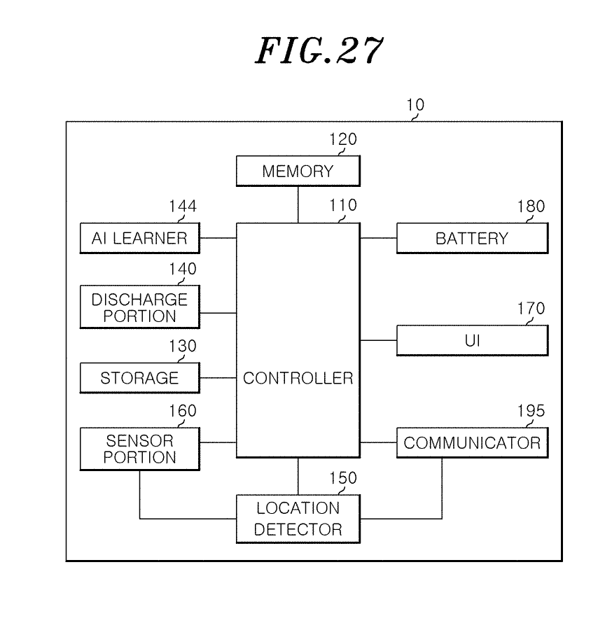

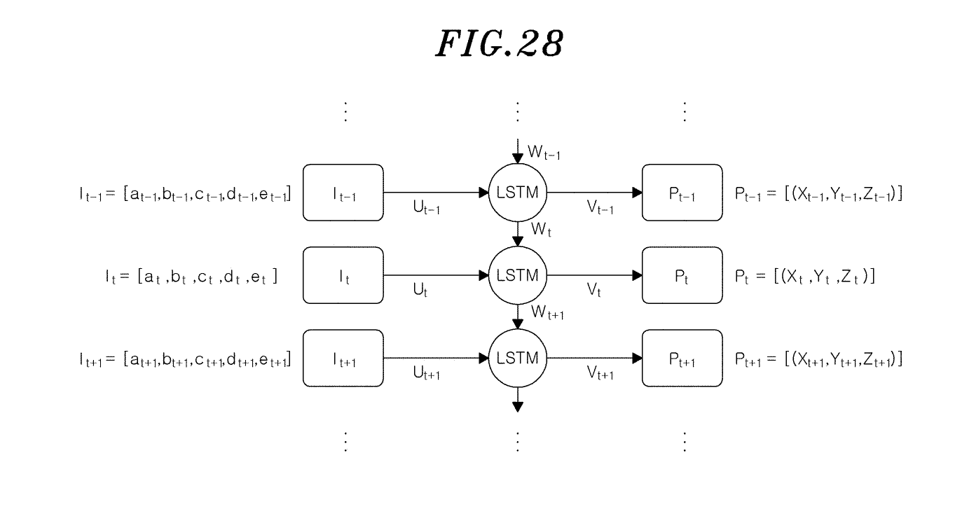

FIG. 27 is a block diagram illustrating a configuration of a mobile fragrance discharge device according to another embodiment, and FIG. 28 illustrates a learning process of an artificial intelligence (AI) learner of FIG. 27.

Referring to FIGS. 27 and 28, the mobile fragrance discharge device according to an embodiment may further include an AI learner 114 in addition to a configuration of the mobile fragrance discharge device shown in FIGS. 1 and 2.

The controller 110 may determine a discharge characteristic of a fragrance material based on location information or travel route information of the body 100, and may control the discharge portion 140 based on the determined discharge characteristic of the fragrance material. Also, the controller 110 may control the discharge portion 140 by further considering at least one of spatial information associated with a space in which the mobile fragrance discharge device 10 is present, state information of the mobile fragrance discharge device 10, and information associated with a person or an object present within the space.

Also, the controller 110 may control the discharge portion 140 by additionally considering spatial information including a spatial characteristic around the mobile fragrance discharge device 10.

The AI learner 114 may learn a control history of the controller 100. For example, the AI learner 114 may learn the control history from the past to the current time on discharging of the fragrance material according to a change in information such as ambient spatial information, information associated with a person or an object present within the space, state information of the mobile fragrance discharge device 10, biometric information of the user, location information and travel route information of the mobile fragrance discharge device 10, time range, space range, and the like while using the mobile fragrance discharge device 10. The control history may be, for example, at least one of a type of a fragrance material, a discharge amount, a discharge pattern, a blending ratio, and a discharge location in the space. The AI learner 114 may generate the control history as a database.

The AI learner may provide a result of learning to the controller 110. Accordingly, the controller 110 may receive, from the AI learner 114, a recommendation on a control condition suitable for an operational situation of the mobile fragrance discharge device 10 to control discharging of the fragrance material. The AI learner 114 may learn the control history from the past to the current time using a time serial data analysis method, for example, a recurrent neural network (RNN).

Also, the AI learner 114 may share the control history on discharging of the fragrance material with respect to a plurality of pieces of user information through a separate external server. The server may use the shared control history data for a time series data analysis.

The RNN may be suitable to learn a change in context over time in terms of AI learning. The RNN may analyze and learn an influence level of a primary factor based on state parameters of a previous time influencing over a current state. FIG. 28 illustrates the RNN. Here, an input vector (Iₜ) refers to context awareness based scent determination (CSD) information and may be spatial information around the mobile fragrance discharge device, information associated with a person or an object present within the space, state information of the mobile fragrance discharge device, biometric information of the user, location information and travel route information of the mobile fragrance discharge device, a time range, and a space rage. Also, an output vector (Pₜ) refers to a CSD value and may be a type, discharge concentration, a discharge location, and a discharge interval of the fragrance material. Also, U, W, and V refer to parameter vectors and may be a recursive method of learning model by adjusting a weight of each parameter and using a result value acquired from the model as an input again. Each cell of the RNN may use a structure such as a long short-term memory (LSTM) and gated recurrent unit (GRU).

Referring to FIG. 28, if I₀ is input, P₁ that is an output value (CSD) in a subsequent time is predicted. The subsequent prediction may be associated with state information of a previous time. Accordingly, P₂ is predicted using an input of I₁ and by adding information of I₀. That is, an RNN learning model has an accumulated value of the previous time and applies context of the previous time. Learning of the RNN is performed to decrease a difference between a predicted value and an actual input value. Here, U, W, and V may be determined using a method of minimizing an error between a prediction value and an actual value of the learning model.

The aforementioned description is provided as an example only and thus, one of ordinary skill in the art to which the disclosure pertains may understand that various modifications may be made without departing from the technical spirit or features of the disclosure. Therefore, the aforementioned embodiments should be understood as examples and construed as not be limiting.

The scope of the disclosure is defined by the claims and the equivalents and all of the changes or modifications from the scope of the claims and the equivalents should be understood to be included in the scope of the disclosure.

What is claimed is:

1. A mobile fragrance discharge device comprising:
a body;
a discharge portion coupled to the body and configured to discharge, to an outside of the body, at least a portion of fragrance material supplied from a storage;
a location detector configured to detect a location or a travel route of the body;
a controller comprising a processing device configured to determine a discharge characteristic of the fragrance material based on location information or travel route information of the body, and to control a fragrance material discharge of the discharge portion based on the determined discharge characteristic; and
a propeller coupled to the body and configured to provide a driving force so that the mobile fragrance discharge device is capable of flight, wherein the storage is configured to be mountable to the body and store the fragrance material.

2. The mobile fragrance discharge device of claim 1, wherein the discharge characteristic comprises at least one of whether to discharge the fragrance material, a type, an amount, a discharge direction, and a discharge pattern of the fragrance material to be discharged, a blending ratio, and a discharge location of the fragrance material on a space.

3. The mobile fragrance discharge device of claim 1, wherein the controller is configured to control the fragrance material discharge of the discharge portion at a predetermined location or in a predetermined section within the travel route.

4. The mobile fragrance discharge device of claim 1, wherein the controller is configured to control the discharge portion by further considering at least one of spatial information associated with a space in which the mobile fragrance discharge device is present, state information of the mobile fragrance discharge device, and information associated with a person or an object present within the space.

5. The mobile fragrance discharge device of claim 1, further comprising:
a sensor portion coupled to the body and configured to sense spatial information comprising a spatial characteristic around the mobile fragrance discharge device,
wherein the controller is configured to control the discharge portion based on the spatial information.

6. The mobile fragrance discharge device of claim 5, wherein the sensor portion comprises at least one of an image sensor, a pollution measurement sensor, a temperature measurement sensor, a fluid velocity sensor, an illuminance sensor, an odor sensor, a location recognition sensor, a proximity sensor, and a sound sensor.

7. The mobile fragrance discharge device of claim 1, wherein the mobile fragrance discharge device is configured to mount to a movable external device and to be movable.

8. The mobile fragrance discharge device of claim 7, further comprising:
a communicator configured to receive the location or the travel route of the body from the mobile external device,
wherein the location detector is configured to detect the location of the body based on the received location information or travel route information.

9. The mobile fragrance discharge device of claim 1, wherein the propeller comprises one or more rotors, and
the mobile fragrance discharge device is a drone capable of moving and hovering in a three-dimensional (3D) space.

10. The mobile fragrance discharge device of claim 1, further comprising:
a communicator configured to interact with an external device and to communicably connect to the external device to control discharging of the fragrance material.

11. The mobile fragrance discharge device of claim 9, wherein the location detector is configured to determine the travel route of the body based on fragrance material related information from the external device.

12. The mobile fragrance discharge device of claim 11, wherein the fragrance material related information comprises at least one of multimedia content information provided from the external device, spatial information comprising a spatial characteristic around the mobile fragrance discharge device, time information, odor information, user body information of a user of the mobile fragrance discharge device, a relative location between the mobile fragrance discharge device and the user, and preference information of the user on a specific fragrance material.

13. The mobile fragrance discharge device of claim 1, wherein the controller is configured to control the propeller to control a travel speed of the mobile fragrance discharge device.

14. The mobile fragrance discharge device of claim 10, wherein the external device is a mobile device, and
a list of external devices operable in conjunction with the mobile fragrance discharge device or a control user interface for controlling the mobile fragrance discharge device is provided to the external device.

15. The mobile fragrance discharge device of claim 10, wherein the external device is another fragrance discharge device, and
the location detector is configured to determine the travel route on a relative location with the other fragrance discharge device and to control discharging of the fragrance material.

16. The mobile fragrance discharge device of claim 10, wherein the location detector is configured to determine the travel route of the body based on fragrance material related information received from the external device, and to determine again the travel route of the body based on new fragrance material related information in response to receiving the new fragrance material related information.

17. The mobile fragrance discharge device of claim 4, wherein the controller is configured to control the discharge portion using a scheme recommended by an Artificial Intelligence (AI) learner, the AI learner configured to learn a control history of the controller on discharging of the fragrance material based on at least one of the spatial information, the state information of the mobile fragrance discharge device, and the information associated with the person or the object present within the space.

18. The mobile fragrance discharge device of claim 18, wherein the AI learner is configured to learn the control history using a time series data analysis.

19. A mobile fragrance discharge device communicably connected to an external device capable of discharging one or more fragrance materials, the mobile fragrance discharge device comprising:
a body;
a discharge portion coupled to the body and configured to discharge, to an outside of the body, fragrance material provided from a storage;
a location detector configured to detect a location or a travel route of the body;
a propeller coupled to the body and configured to provide a driving force so that the mobile fragrance discharge device is capable of flight;
a communicator configured to communicably connect to the external device; and
a controller comprising a processing device configured to determine a discharge characteristic of the fragrance material based on location information or travel route information of the body, and to control the discharge portion to discharge the fragrance material based on the determined discharge characteristic,
wherein the controller is configured to control a fragrance material discharge of the external device through the communicator, wherein the storage is configured to be mountable to the body and store the fragrance material.

20. A mountable fragrance container comprising:
a storage configured to be mountable to a body and store fragrance material, wherein:
the body is coupled to a discharge portion configured to discharge, to an outside of the body, at least a portion of the fragrance material supplied from the storage,
the storage is further configured to be controlled by a controller, the controller comprising a processing device configured to determine a discharge characteristic of the fragrance material based on location information or travel route information of the body, and further configured to control a fragrance material discharge of the discharge portion based on the determined discharge characteristic, and
the body is further coupled to a propeller, the propeller configured to provide a driving force so that the body is capable of flight.
